(19) United States
(12) Patent Application Publication
(10) Pub. No.: US 2022/0176120 A1
(43) Pub. Date: Jun. 9, 2022

(54) SYSTEMS WITH IMPLANTED CONDUIT TRACKING

(71) Applicant: Nalu Medical, Inc., Carlsbad, CA (US)

(72) Inventors: Gaurav Gajanan KULKARNI, Pune (IN); Udayan KANADE, Pune (IN); Lakshmi Narayan MISHRA, Carlsbad, CA (US)

(21) Appl. No.: 17/383,972

(22) Filed: Jul. 23, 2021

Related U.S. Application Data
(63) Continuation of application No. PCT/US20/66901, filed on Dec. 23, 2020.
(60) Provisional application No. 62/952,717, filed on Dec. 23, 2019.

(51) Int. Cl. A61N 1/36; A61N 1/372; A61N 1/378; A61N 1/05

(52) U.S. Cl. CPC A61N 1/3614; A61N 1/36071; A61N 1/05; A61N 1/37235; A61N 1/3787; A61N 1/37229

(57) ABSTRACT

A medical apparatus is provided comprising a delivery device and an algorithm. The delivery device comprises: a plurality of electrodes, a first lead, and a second lead. The plurality of electrodes comprises a first set of electrodes comprising one or more electrodes, and a second set of electrodes comprising one or more electrodes. The first lead comprises the first set of electrodes, and the second lead comprises the second set of electrodes. The delivery device is configured to measure impedance between multiple pairs of electrodes of the plurality of electrodes. The algorithm is configured to determine position information of the first lead and/or the second lead.

### CROSS-REFERENCE

This application is a continuation of PCT Application No. PCT/US20/66901, filed Dec. 23, 2020; which claims priority to U.S. Provisional Application No. 62/952,717, filed Dec. 23, 2019; the contents of which are incorporated herein by reference in their entirety for all purposes.

### RELATED APPLICATIONS

This application is related to: U.S. patent application Ser. No. 14/975,358, titled “Method and Apparatus for Minimally Invasive Implantable Modulators”, filed Dec. 18, 2015 [Docket nos. 47476.703.301; NAL-005-US]; U.S. patent application Ser. No. 15/664,231, titled “Medical Apparatus Including an Implantable System and an External System”, filed Jul. 31, 2017 [Docket nos. 47476-706.301; NAL-011-US]; U.S. patent application Ser. No. 16/104,829, titled “Apparatus with Enhanced Stimulation Waveforms”, filed Aug. 17, 2018 [Docket nos. 47476-708.301; NAL-014-US]; U.S. patent application Ser. No. 16/111,868, titled “Devices and Methods for Positioning External Devices in Relation to Implanted Devices”, filed Aug. 24, 2018 [Docket nos. 47476-709.301; NAL-016-US]; U.S. patent application Ser. No. 16/222,959, titled “Methods and Systems for Treating Pelvic Disorders and Pain Conditions”, filed Dec. 17, 2018 [Docket nos. 47476-711.301; NAL-017-US]; U.S. patent application Ser. No. 16/266,822, titled “Method and Apparatus for Versatile Minimally Invasive Neuromodulators”, filed Feb. 4, 2019 [Docket nos. 47476-704.302; NAL-007-US-CON1]; U.S. patent application Ser. No. 16/408,989, titled “Method and Apparatus for Neuromodulation Treatments of Pain and Other Conditions”, filed May 10, 2019 [Docket nos. 47476.705.302; NAL-008-US-CON1]; U.S. patent application Ser. No. 16/453,917, titled “Stimulation Apparatus”, filed Jun. 26, 2019 [Docket nos. 47476-712.301; NAL-015-US]; U.S. patent application Ser. No. 16/505,425, titled “Wireless Implantable Sensing Devices”, filed Jul. 8, 2019 [Docket nos. 10220-728.300; NAL-006-US-CON1]; U.S. patent application Ser. No. 16/539,977, titled “Apparatus with Sequentially Implanted Stimulators”, filed Aug. 13, 2019 [Docket nos. 47476-713.301; NAL-019-US]; U.S. patent application Ser. No. 16/672,921, titled “Stimulation Apparatus”, filed Nov. 4, 2019 [Docket nos. 47476-714.301; NAL-020-US]; U.S. Provisional Application Ser. No. 63/042,293, titled “Systems with Implanted Conduit Tracking”, filed Jun. 22, 2020 [Docket nos. 47476-717.101; NAL-023-PR1]; International PCT Patent Application Serial Number PCT/US2020/040766, titled “Stimulation Apparatus”, filed Jul. 2, 2020 [Docket nos. 47476-715.601; NAL-021-PCT]; U.S. patent application Ser. No. 16/993,999, titled “Apparatus for Peripheral or Spinal Stimulation”, filed Aug. 14, 2020 [Docket nos. 47476-707.302; NAL-012-US-CON1]; U.S. Provisional Application Ser. No. 63/071,925, titled “Apparatus for Delivering Customized Stimulation Waveforms”, filed Aug. 28, 2020 [Docket Nos. 47476-718.101; NAL-024-PR1]; U.S. Provisional Application Ser. No. 63/082,856, titled “Stimulation Energy Systems with Current Steering”, filed Sep. 24, 2020 [Docket Nos. 47476-717.102; NAL-023-PR2]; International PCT Patent Application Serial Number PCT/US2020/054150, titled “Stimulation Apparatus”, filed Oct. 2, 2020 [Docket Nos. 47476-719.601; NAL-025-PCT]; and U.S. patent application Ser. No. 17/081,351, titled “Methods and Systems for Insertion and Fixation of Implantable Devices”, filed Oct. 27, 2020 [Docket nos. 47476-710.302; NAL-013-US-CON1]; the contents of each of which is incorporated herein by reference in its entirety for all purposes.

### TECHNICAL FIELD

The present invention relates generally to medical apparatus for a patient, and in particular, systems including implantable conduits whose implant location or migration from that location can be tracked.

### BACKGROUND OF THE INVENTION

Delivery devices that treat a patient and/or record patient data are known. For example, implants and other delivery devices that deliver energy such as electrical energy, or deliver agents such as pharmaceutical agents are commercially available. Electrical stimulators can be used to pace or defibrillate the heart, as well as modulate nerve tissue (e.g. to treat pain). Most implants are relatively large devices with batteries and long conduits, such as implantable leads configured to deliver electrical energy or implantable tubes (i.e. catheters) to deliver an agent. These implants require a fairly invasive implantation procedure, and periodic battery replacement, which requires additional surgery. The large sizes of these devices and their high costs have prevented their use in a variety of applications.

Nerve stimulation treatments have shown increasing promise recently, showing potential in the treatment of many chronic diseases including drug-resistant hypertension, motility disorders in the intestinal system, metabolic disorders arising from diabetes and obesity, and both chronic and acute pain conditions among others. Many of these delivery device configurations have not been developed effectively because of the lack of miniaturization and power efficiency, in addition to other limitations. For example, migration of implanted components can lead to compromised results.

There is a need for apparatus, systems, devices and methods that provide one or more therapy delivering devices and are designed to provide enhanced therapy and other enhanced benefits.

### SUMMARY

According to an aspect of the present inventive concepts, a medical apparatus for a patient comprises a delivery device and an algorithm. The delivery device comprises: a plurality of electrodes, a first lead, and a second lead. The plurality of electrodes comprises a first set of electrodes comprising one or more electrodes, and a second set of electrodes comprising one or more electrodes. The first lead comprises the first set of electrodes, and the second lead comprises the second set of electrodes. The delivery device is configured to measure impedance between multiple pairs of electrodes of the plurality of electrodes. The algorithm is configured to determine position information of the first lead and/or the second lead based on the measured impedances.

In some embodiments, the position information comprises angular rotation information of the first lead and/or the second lead. The position information can comprise angular rotation information of the first lead and the second lead.

In some embodiments, the position information comprises the position of the first lead and/or the second lead relative to the patient's anatomy.

In some embodiments, the position information comprises the position of the first lead relative to the position of the second lead.

In some embodiments, the position information comprises the position of the first lead relative to the patient's anatomy at a first instance of time, compared to the position of the first lead relative to the patient's anatomy at a second instance of time, and the second instance of time is previous to the first instance in time.

In some embodiments, the position information comprises the position of the first lead relative to the second lead at a first instance of time, compared to the position of the first lead relative to the second lead at a second instance of time, and the second instance of time is previous to the first instance in time.

In some embodiments, the algorithm comprises a mathematical model and a list of pairs of electrodes selected from the plurality of electrodes, and the algorithm determines the position information based on measured impedances that best fit the mathematical model.

In some embodiments, the algorithm is based on data gathered prior to implantation of the delivery device in the patient. The data can be gathered during the manufacturing of the delivery device.

In some embodiments, the algorithm is configured to determine a relative position between the first lead and the second lead by: (1) measuring the impedance between at least one pair of electrodes of the first set of electrodes and at least one pair of electrodes of the second set of electrodes; (2) fitting a curve to the measured impedances to obtain a function of the impedance to distance: Z=f(d), based on the known distances between the electrodes of each pair; (3) measuring the impedance between at least one cross-lead pair of electrodes, each cross-lead pair comprising one electrode of the first set of electrodes and one electrode of the second set of electrodes; (4) determining the distance between the at least one cross-lead pair of electrodes using the function of (2); (5) determining the relative positions of the first lead and the second lead using the calculated distances. The at least one pair of electrodes of the first set of electrodes can comprise all pairs of electrodes of the first set of electrodes, and the at least one pair of electrodes of the second set of electrodes can comprise all pairs of electrodes of the second set of electrodes. The impedance measurements can include at least 56 impedance measurements per lead. The at least one cross-lead pair of electrodes can comprise at least 64 pairs of electrodes. The relative position can include a first linear offset Lx, a second linear offset Ly, and/or an angle θ between the first lead and the second lead.

In some embodiments, the algorithm is configured to determine a relative position between the first lead and the second lead by: (1) measuring the impedance between at least one pair of electrodes of the first set of electrodes and at least one pair of electrodes of the second set of electrodes; (2) creating a first resistivity profile of tissue surrounding the first lead and creating a second resistivity profile of tissue surrounding the second lead based on the impedance measurements; (3) measuring the impedance between at least one cross-lead pair of electrodes, each cross-lead pair comprising one electrode of the first set of electrodes and one electrode of the second set of electrodes; (4) determining the distance between the at least one cross-lead pair of electrodes using a linear resistivity assumption based on the first resistivity profile and the second resistivity profile; (5) determining the relative positions of the first lead and the second lead using the calculated distances. The at least one pair of electrodes of the first set of electrodes can comprise all pairs of electrodes of the first set of electrodes, and the at least one pair of electrodes of the second set of electrodes can comprise all pairs of electrodes of the second set of electrodes. The impedance measurements can include at least 56 impedance measurements per lead. The at least one cross-lead pair of electrodes can comprise at least 64 pairs of electrodes. The relative position can include a first linear offset Lx, a second linear offset Ly, and/or an angle θ between the first lead and the second lead.

In some embodiments, the algorithm is configured to characterize a migration of the first lead and/or second lead by: (1) determining the relative positions of the first lead and the second lead at a first time T1; (2) creating an initial graph based on the relative positions at the first time T1; (3) determining the relative positions of the first lead and the second lead at a second time T2; (4) creating a subsequent graph based on the relative positions at the second time T2; (5) determining the difference between the initial graph and the subsequent graph to determine the migration of the first lead and/or the second lead between the first time T1 and the second time T2. The relative positions of the first lead and the second lead can be determined using a resistivity profile. The relative positions of the first lead and the second lead can be determined using impedance measurements. The migration of the first lead and the second lead can comprise a relative linear migration between the first lead and the second lead.

In some embodiments, the algorithm comprises one or more equations comprising the measured impedances, and the algorithm can be configured to determine a relative position between the first lead and second lead by: (1) measuring the impedance between at least one pair of electrodes of the first set of electrodes and at least one pair of electrodes of the second set of electrodes; (2) measuring the impedance between at least one cross-lead pair of electrodes, each cross-lead pair comprising one electrode of the first set of electrodes and one electrode of the second set of electrodes; (3) determining resistivities of layers of the body, a bias impedance, and the relative position between the first lead and second lead so as to minimize errors in the one or more equations comprising the measured impedances. Each equation of the one or more equations can equate a measured impedance to the sum of the bias impedance and a compound term. The compound term can be a sum of a plurality of products, and each product in the plurality of products can comprise a resistivity of one layer of the body, a length of a line segment, and a weight. The line segment can be the intersection of a line connecting the pair of electrodes across which the measured impedance can be measured and the layer of the body. The weight can be calculated using a weighing function of length. The relative position of the first lead and the second lead can comprise a relative vertical displacement between the first lead and the second lead. The relative position of the first lead and the second lead can comprise a relative horizontal displacement between the first lead and the second lead. The relative position of the first lead and the second lead can comprise a relative angular displacement between the first lead and the second lead.

In some embodiments, the algorithm comprises multiple algorithms.

In some embodiments, the delivery device further comprises a power supply, a controller, and a housing surrounding the power supply and the controller, and the first lead and/or the second lead is pre-attached to the housing.

In some embodiments, the delivery device further comprises a power supply, a controller, and a housing surrounding the power supply and the controller, and the first lead and/or the second lead is attachable to the housing during a clinical procedure in which the delivery device is implanted in the patient.

In some embodiments, the first lead and/or the second lead further comprise at least one functional element. The at least one functional element can comprise one or more stimulation elements configured to deliver therapy to the patient, and the therapy can comprise delivery of electrical energy to tissue of the patient. The at least one functional element can comprise one or more stimulation electrodes, and the plurality of electrodes can comprise the one or more stimulation electrodes. The at least one functional element can comprise one or more therapy delivery elements configured to delivery therapy to the patient. The one or more therapy delivery elements can be configured to deliver therapy to tissue comprising: light energy; laser light energy; sound energy; ultrasound energy; an agent; and combinations thereof. The at least one functional element can comprise one or more sensors configured to record physiologic information of the patient.

In some embodiments, one or more of the plurality of electrodes comprises a coating and/or a surface finish configured to lower the impedance of the associated electrode.

In some embodiments, the first set of electrodes comprises at least four electrodes and the second set of electrodes comprises at least four electrodes. The first set of electrodes can comprise at least six electrodes and the second set of electrodes can comprise at least six electrodes. The first set of electrodes can comprise at least eight electrodes and the second set of electrodes can comprise at least eight electrodes.

In some embodiments, the first set of electrodes and/or the second set of electrodes comprise a set of three or more electrodes that are separated by the same separation distance.

In some embodiments, the first set of electrodes and/or the second set of electrodes comprise a set of three or more electrodes that are separated by different separation distances.

In some embodiments, the apparatus is configured to provide therapy to the patient. The apparatus can be configured to treat pain of the patient.

In some embodiments, the apparatus is configured to record physiologic information of the patient.

In some embodiments, the apparatus can further comprise an external system configured to transmit power and/or data to the delivery device.

In some embodiments, the delivery device further comprises a power supply, a controller, and a housing surrounding the power supply and the controller, and the housing comprises at least a portion that is configured as an electrode.

In some embodiments, the delivery device further comprises a power supply. The power supply can comprise a battery and/or a capacitor. The power supply can be configured to receive power from an external source.

According to another aspect of the present inventive concepts, a method of detecting the position of an implanted lead comprises: providing an apparatus as described herein; providing a list of pairs of electrodes of the plurality of electrodes; measuring impedance across each pair of electrodes from the list of pairs of electrodes; and determining parameters of a mathematical model to create a best match to the measured impedances.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view of a medical apparatus comprising a delivery device with an implantable lead, consistent with the present inventive concepts.

FIG. 1A is a schematic anatomical view of a medical apparatus comprising an external system and an implantable system, consistent with the present inventive concepts.

FIG. 2 is a schematic view of two leads, consistent with the present inventive concepts.

FIGS. 3A-B are two schematic views of a pair of leads that have been implanted in a patient, consistent with the present inventive concepts.

FIG. 4 is a graph representing migration of an implanted lead, consistent with the present inventive concepts.

FIG. 5 is a graph of variation in the value of cross-correlation for different values of shifts in implanted leads, consistent with the present inventive concepts.

FIG. 6 is a schematic view of a pair of leads modeled with a layered construction of the patient's body, consistent with the present inventive concepts.

### DETAILED DESCRIPTION OF THE DRAWINGS

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers, and/or sections, these limitations, elements, components, regions, layers, and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.). A first component (e.g. a device, assembly, housing or other component) can be “attached”, “connected” or “coupled” to another component via a connecting filament (as defined below). In some embodiments, an assembly comprising multiple components connected by one or more connecting filaments is created during a manufacturing process (e.g. pre-connected at the time of an implantation procedure of the apparatus of the present inventive concepts). Alternatively or additionally, a connecting filament can comprise one or more connectors (e.g. a connectorized filament comprising a connector on one or both ends), and a similar assembly can be created by a user (e.g. a clinician) operably attaching the one or more connectors of the connecting filament to one or more mating connectors of one or more components of the assembly.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of these.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

As used herein, the term “proximate” shall include locations relatively close to, on, in, and/or within a referenced component or other location.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

The term “functional element” where used herein, is the be taken to include a component comprising one, two or more of: a sensor; a transducer; an electrode; an energy delivery element; an agent delivery element; a magnetic field generating transducer; and combinations of these. In some embodiments, a functional element comprises a transducer selected from the group consisting of: light delivery element; light emitting diode; wireless transmitter; Bluetooth device; mechanical transducer; piezoelectric transducer; pressure transducer; temperature transducer; humidity transducer; vibrational transducer; audio transducer; speaker; and combinations of these. In some embodiments, a functional element comprises a needle, a catheter (e.g. a distal portion of a catheter), an iontophoretic element or a porous membrane, such as an agent delivery element configured to deliver one or more agents. In some embodiments, a functional element comprises one or more sensors selected from the group consisting of: electrode; sensor configured to record electrical activity of tissue; blood glucose sensor such as an optical blood glucose sensor; pressure sensor; blood pressure sensor; heart rate sensor; inflammation sensor; neural activity sensor; muscular activity sensor; pH sensor; strain gauge; accelerometer; gyroscope; GPS; respiration sensor; respiration rate sensor; temperature sensor; magnetic sensor; optical sensor; MEMs sensor; chemical sensor; hormone sensor; impedance sensor; tissue impedance sensor; body position sensor; body motion sensor; physical activity level sensor; perspiration sensor; patient hydration sensor; breath monitoring sensor; sleep monitoring sensor; food intake monitoring sensor; urine movement sensor; bowel movement sensor; tremor sensor; pain level sensor; orientation sensor; motion sensor; and combinations of these.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy, mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); thermal energy to tissue (e.g. heat energy and/or cryogenic energy); chemical energy; electromagnetic energy; magnetic energy; and combinations of these.

The term “transmission signal” where used herein is to be taken to include any signal transmitted between two components, such as via a wired or wireless communication pathway. For example, a transmission signal can comprise a power and/or data signal wirelessly transmitted between a component external to the patient and one or more components implanted in the patient. A transmission signal can include one or more signals transmitted using body conduction. Alternatively or additionally, a transmission signal can comprise reflected energy, such as energy reflected from any power and/or data signal.

The term “data signal” where used herein is to be taken to include a transmission signal including at least data. For example, a data signal can comprise a transmission signal including data and sent between a component external to the patient and one or more components implanted in the patient. Alternatively, a data signal can comprise a transmission signal including data sent from an implanted component to one or more components external to the patient. A data signal can comprise a radiofrequency signal including data (e.g. a radiofrequency signal including both power and data) and/or a data signal sent using body conduction.

The term “implantable” where used herein is to be taken to define a component which is constructed and arranged to be fully or partially implanted in a patient's body and/or a component that has been fully or partially implanted in a patient. The term “external” where used herein is to be taken to define a component which is constructed and arranged to be positioned outside of the patient's body.

The terms “attachment”, “attached”, “attaching”, “connection”, “connected”, “connecting” and the like, where used herein, are to be taken to include any type of connection between two or more components. The connection can include an “operable connection” or “operable attachment” which allows multiple connected components to operate together such as to transfer information, power, and/or material (e.g. an agent to be delivered) between the components. An operable connection can include a physical connection, such as a physical connection including a connection between two or more: wires or other conductors (e.g. an “electrical connection”), optical fibers, wave guides, tubes such as fluid transport tubes, and/or linkages such as translatable rods or other mechanical linkages. Alternatively or additionally, an operable connection can include a non-physical or “wireless” connection, such as a wireless connection in which information and/or power is transmitted between components using electromagnetic energy. A connection can include a connection selected from the group consisting of: a wired connection; a wireless connection; an electrical connection; a mechanical connection; an optical connection; a sound propagating connection; a fluid connection; and combinations of these.

The term “connecting filament” where used herein is to be taken to define a filament connecting a first component to a second component. The connecting filament can include a connector on one or both ends, such as to allow a user to operably attach at least one end of the filament to a component. A connecting filament can comprise one or more elements selected from the group consisting of: wires; optical fibers; fluid transport tubes; mechanical linkages; wave guides; flexible circuits; and combinations of these. A connecting filament can comprise rigid filament, a flexible filament or it can comprise one or more flexible portions and one or more rigid portions.

The term “connectorized” where used herein is to be taken to refer to a filament, housing or other component that includes one or more connectors (e.g. clinician or other user-attachable connectors) for operably connecting that component to a mating connector (e.g. of the same or different component).

The terms “stimulation parameter”, “stimulation signal parameter” or “stimulation waveform parameter” where used herein can be taken to refer to one or more parameters of a stimulation waveform (also referred to as a stimulation signal). Applicable stimulation parameters of the present inventive concepts shall include but are not limited to: amplitude (e.g. amplitude of voltage and/or current); average amplitude; peak amplitude; frequency; average frequency; pulse width (also referred to as “pulse pattern on time”); period; phase; polarity; pulse shape; a duty cycle parameter (e.g. frequency, pulse width, and/or off time); inter-pulse gap (also referred to as “pulse pattern off time”, or “inter-pulse interval”); polarity; burst-on (also referred to as “dosage on”) period; burst-off (also referred to as “dosage off”) period; inter-burst period; pulse train; train-on period; train-off period; inter-train period; drive impedance; duration of pulse and/or amplitude level; duration of stimulation waveform; repetition of stimulation waveform; an amplitude modulation parameter; a frequency modulation parameter; a burst parameter; a power spectral density parameter; an anode/cathode configuration parameter; amount of energy and/or power to be delivered; rate of energy and/or power delivery; time of energy delivery initiation; method of charge recovery; and combinations of these. A stimulation parameter can refer to a single stimulation pulse, multiple stimulation pulses, or a portion of a stimulation pulse. The term “amplitude” where used herein can refer to an instantaneous or continuous amplitude of one or more stimulation pulses (e.g. the instantaneous voltage level or current level of a pulse). The term “pulse” where used herein can refer to a period of time during which stimulation energy is relatively continuously being delivered. In some embodiments, stimulation energy delivered during a pulse comprises energy selected from the group consisting of: electrical energy; magnetic energy; electromagnetic energy; light energy; sound energy such as ultrasound energy; mechanical energy such as vibrational energy; thermal energy such as heat energy or cryogenic energy; chemical energy; and combinations of these. In some embodiments, stimulation energy comprises electrical energy and a pulse comprises a phase change in current and/or voltage. In these embodiments, an “inter-phase gap” can be present within a single pulse. The term inter-phase gap where used herein can refer to a period of time between two portions of a pulse comprising a phase change during which zero energy or minimal energy is delivered. The term “quiescent period” where used herein can refer to a period of time during which zero energy or minimal energy is delivered (e.g. insufficient energy to elicit an action potential and/or other neuronal response). The term “inter-pulse gap” where used herein can refer to a quiescent period between the end of one pulse to the onset of the next (sequential) pulse. The terms “pulse train” or “train” where used herein can refer to a series of pulses. The terms “burst”, “burst of pulses” or “burst stimulation” where used herein can refer to a series of pulse trains, each separated by a quiescent period. The term “train-on period” where used herein can refer to a period of time from the beginning of the first pulse to the end of the last pulse of a single train. The term “train-off period” where used herein can refer to a quiescent period between the end of one train and the beginning of the next train. The term “burst-on period” where used herein can refer to a period of time from the beginning of the first pulse of the first train to the end of the last pulse of the last train of a single burst. The term “burst-off period” where used herein can refer to a quiescent period between the end of one burst and the beginning of the next burst. The term “inter-train period” where used herein can refer to a quiescent period between the end of one train and the beginning of the next train. The term “inter-burst period” where used herein can refer to a quiescent period between the end of one burst and the beginning of the next burst. The term “train envelope” where used herein can refer to a curve outlining the amplitude extremes of a series of pulses in a train. The term “burst envelope” where used herein can refer to a curve outlining the amplitude extremes of a series of pulses in a burst. The term “train ramp duration” where used herein can refer to the time from the onset of a train until its train envelope reaches a desired target magnitude. The term “burst ramp duration” where used herein can refer to the time from the onset of a burst until its burst envelope reaches a desired target magnitude.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

The present inventive concepts include a medical apparatus and clinical methods for treating a medical condition of a patient, such as a disease or disorder of the patient. Alternatively or additionally, the medical apparatus can be configured for performing a diagnostic and/or prognostic (“diagnostic” herein) procedure on a patient. The patient can comprise a human or other mammalian patient. The medical apparatus can comprise a stimulation or other therapy-providing apparatus. The medical apparatus can comprise an implantable system and an external system. The implantable system can comprise one or more similar and/or dissimilar delivery devices. Each delivery device comprises a housing surrounding one or more therapy-providing components (e.g. stimulation producing components) and/or sensing components. One or more leads (e.g. flexible leads) can be pre-attached to the housing, or the leads can be attachable to the housing (e.g. attached in a clinical procedure in which at least the distal portion of the lead of the delivery device is implanted in a patient).

Each lead can comprise one or more therapy-providing functional elements, such as elements configured to delivery stimulation energy (e.g. electrical, light, and/or sound energy) and/or elements configured to deliver an agent (e.g. a pharmaceutical drug or other agent). Alternatively or additionally, each lead can comprise one or more sensors, such as one or more physiologic sensors. Each lead can comprise one or more electrodes configured to identify the position or change in position of the lead (e.g. position or change in position of the lead as implanted in the patient). In some embodiments, these position-identifying electrodes are further configured as a functional element that provides the therapeutic stimulation energy (e.g. electrical energy).

Each delivery device can comprise one or more antennas configured to receive power and/or data. Each delivery device can comprise a receiver configured to receive the power and/or data from the one or more antennas. Each delivery device can comprise one or more functional elements (e.g. an implantable stimulation element). A functional element of a delivery device can be configured to interface with the patient (e.g. interface with tissue of the patient or interface with any patient location). Alternatively or additionally, a functional element of a delivery device can interface with a portion of its delivery device (e.g. to measure a delivery device parameter). In some embodiments, one or more functional elements of a delivery device can comprise one or more transducers, electrodes, and/or other elements configured to deliver energy to tissue. Alternatively or additionally, one or more functional elements of a delivery device can comprise one or more sensors, such as a sensor configured to record a physiologic parameter of the patient. In some embodiments, one or more functional elements of a delivery device are configured to record device information and/or patient information (e.g. patient physiologic or patient environment information).

Each delivery device can comprise a controller configured to control (e.g. modulate power to, send a signal to, and/or receive a signal from) the one or more functional elements of the delivery device. In some embodiments, a controller of a first delivery device is configured to control one or more other delivery devices (e.g. one or more other delivery device that have been implanted in the patient). Each delivery device can comprise an energy storage assembly (e.g. a battery and/or a capacitor) configured to provide power to the controller (e.g. a controller comprising a stimulation waveform generator), the receiver and/or the one or more functional elements of the delivery device. In some embodiments, an energy storage assembly is further configured to provide power to an assembly that transmits signals via the antenna of the delivery device (e.g. when the delivery device is further configured to transmit data to one or more other devices of the apparatus). Each delivery device can comprise a housing (e.g. an implantable housing) surrounding the controller and the receiver. In some embodiments, one or more antennas are positioned within the housing of the delivery device. Alternatively or additionally, one or more antennas and/or functional elements can be tethered (e.g. electrically tethered) to the housing of the delivery device. In some embodiments, one or more functional elements are positioned on an implantable lead, such as a flexible lead mechanically fixed or attachable to the delivery device housing and operably connected (e.g. electrically, fluidly, optically and/or mechanically) to one or more components internal to the housing. The implantable lead can be inserted (e.g. tunneled) through tissue of the patient, such that its one or more functional elements are positioned proximate tissue to be treated and/or positioned at an area in which data is to be recorded. In some embodiments, the implantable lead is configured to operably attach to and/or detach from, multiple delivery devices.

The external system of the medical apparatus of the present inventive concepts can comprise one or more similar and/or dissimilar external devices. Each external device can comprise one or more external antennas configured to transmit power and/or data to one or more implanted components of the implantable system. Each external device can comprise an external transmitter configured to drive the one or more external antennas. Each external device can comprise an external power supply configured to provide power to at least the external transmitter. Each external device can comprise an external programmer configured to control the external transmitter and/or an implantable device (e.g. when an external power transmitter is not included in the apparatus or otherwise not present during use). Each external device can comprise an external housing that surrounds at least the external transmitter. In some embodiments, the external housing surrounds the one or more external antennas, the external power supply and/or the external programmer.

The external programmer can comprise a discrete controller separate from the one or more external devices, and/or a controller integrated into one or more external devices. The external programmer can comprise a user interface, such as a user interface configured to set and/or modify one or more treatment and/or data recording settings of the medical apparatus of the present inventive concepts. In some embodiments, the external programmer is configured to collect and/or diagnose recorded patient information, such as to provide the information and/or diagnosis to a clinician of the patient, to a patient family member and/or to the patient themselves. The collected information and/or diagnosis can be used to adjust treatment or other operating parameters of the medical apparatus. In some embodiments, at least two external programmers are included, such as a first external programmer configured for use by the patient, and a second external programmer configured for use by a clinician of the patient.

In some embodiments, a medical apparatus comprises a stimulation apparatus for activating, blocking, affecting or otherwise stimulating (hereinafter “stimulate” or “stimulating”) tissue of a patient, such as nerve tissue or nerve root tissue (hereinafter “nerve”, “nerves”, “nerve tissue” or “nervous system tissue”). The stimulation apparatus comprises an external system configured to transmit power, and an implanted system configured to receive the power from the external system and to deliver therapy (e.g. deliver stimulation energy and/or an agent to tissue). Therapy comprising delivered stimulation energy can comprise one or more stimulation waveforms, such as a stimulation waveform configured to enhance treatment of pain while minimizing undesired effects. The stimulation signal (also referred to as “stimulation energy” herein) delivered by the implanted system can be independent of the power received from the external system, such as to be independent of one or more of: the position of one or more components of the external system; the changing position of one or more components of the external system; the frequency of the power received from the external system; the amplitude of the power received from the external system; changes in amplitude of the power received from the external system; duty cycle of the power received from the external system; envelope of the power received from the external system; and combinations of these.

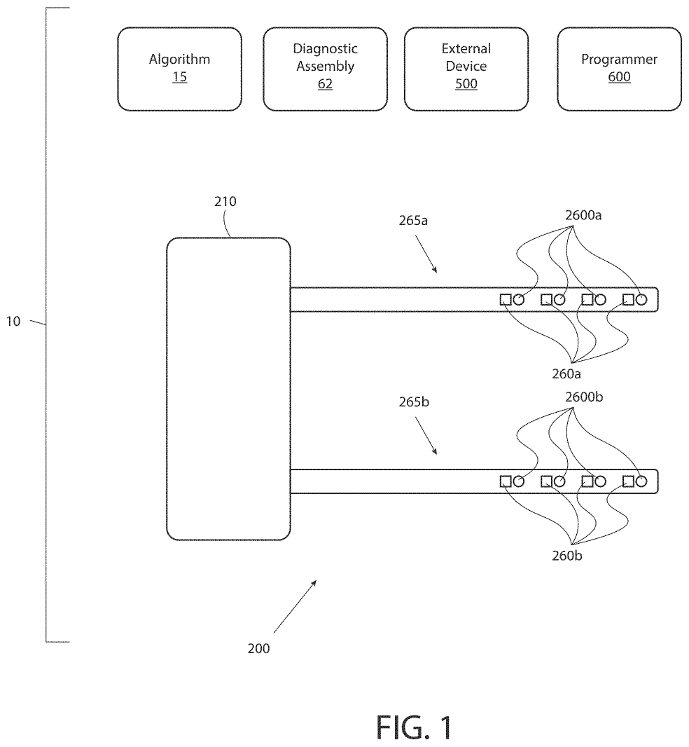

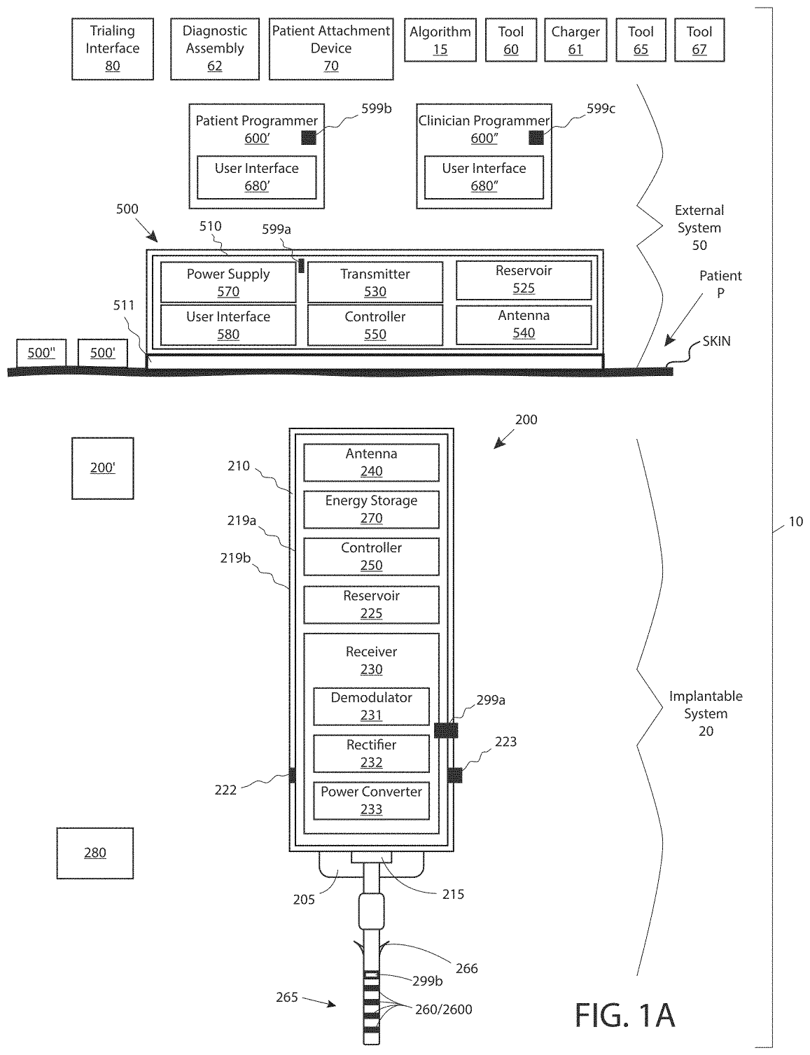

Referring now to FIG. 1, a schematic view of a medical apparatus for a patient is illustrated, consistent with the present inventive concepts. Apparatus 10 comprises delivery device 200, which includes one or more leads, lead 265 (two shown, 265a and 265b, in FIG. 1), which extends from a housing, housing 210. Delivery device 200 can be configured to provide a therapy to a patient (e.g. stimulation therapy), and/or to record patient information, such as patient physiologic information. At least a portion (e.g. at least the distal portion) of leads 265 can be configured to be implanted in a patient (i.e. positioned under the skin of the patient). In some embodiments, housing 210 is also configured for implantation in the patient (e.g. when delivery device 200 is implanted in the patient in its entirety). Apparatus 10 can comprise one or more algorithms, algorithm 15 shown. Algorithm 15 can be configured to determine location information regarding one or more leads 265, as described herein. In some embodiments, algorithm 15 comprises one or more mathematical models. In these embodiments, algorithm 15 can analyze measured impedances between pairs of electrodes (e.g. electrodes on two or more leads 265), and determine parameters of a mathematical model to create a best match to the measured impedances, as described herein.

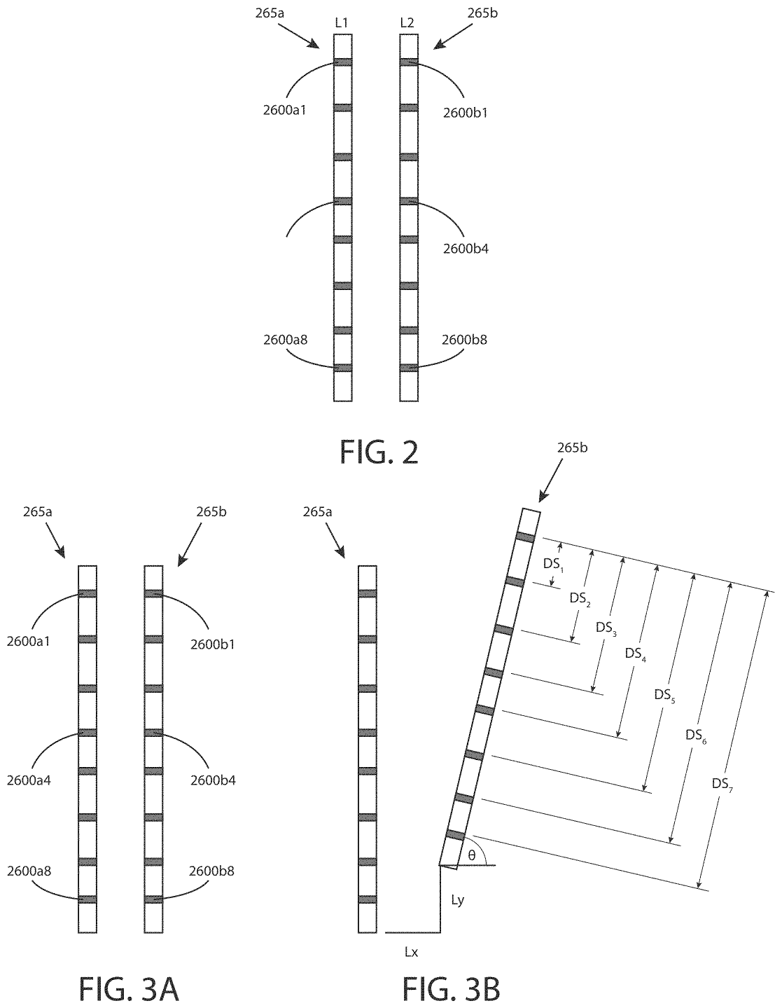

Each lead 265 can comprise one or more electrodes 2600 (four shown for lead 265a and four shown for lead 265b). In some embodiments, a single lead 265 comprises 1, 2, 3, 4, 6, and/or 8 electrodes 2600. Each electrode 2600 can comprise a component configured to deliver current (also referred to as “source current” herein) and/or receive current (also referred to as “sink current” herein). Current transmitted between two or more electrodes 2600 (via tissue in between the two or more electrodes 2600) can be used by apparatus 10 (e.g. used by algorithm 15) to identify (e.g. provide information related to) one or more of the following: the current position of one or more leads 265; the change in position of one or more leads 265 (e.g. the change in position between two or more instances in time); the relative position between two or more leads 265; the change in the relative position between two or more leads 265 (e.g. the change in position between two or more instances in time); and combinations of these, such as is described herein. In some embodiments, algorithm 15 is configured to provide lead 265 location information as described in reference to FIGS. 2-6 herein.

In some embodiments, electrodes 2600 comprise electrodes with a length of 0.5 mm, a length of 3.0 mm, or any length in between. Electrodes 2600 can comprise an electrode with an outer diameter of 1.35 mm. Electrodes 2600 can comprise electrodes constructed of platinum and iridium, such as platinum and iridium at a 9:1 ratio. Sets of electrodes 2600 positioned on a single lead 265 can be separated 0.5 mm apart from each other, 4.0 mm apart from each other, or at a separation distance between 0.5 mm and 4.0 mm. Electrodes 2600 can comprise electrodes with one or more coatings and/or finishes, such as a coating or a finish that reduces the impedance of the electrode and/or increases the surface area of the electrode.

In some embodiments, lead 265 comprises a catheter (e.g. a single or multi-lumen catheter) configured to deliver a pharmaceutical drug or other therapeutic agent (e.g. an agent stored within a reservoir of delivery device 200, not shown). Alternatively or additionally, delivery device 200 comprises one or more functional elements configured to provide therapy and/or perform a diagnostic function, such as stimulation elements 260 (four shown for lead 265a and four shown for lead 265b). In some embodiments, one or more stimulation elements 260, and a corresponding electrode 2600, are the same component (i.e. the same electrode), such as when the one or more stimulation elements comprise an electrode configured to deliver stimulation energy in the form of electrical energy. Alternatively or additionally, one or more stimulation elements 260 can comprise a non-electrical energy delivering component (e.g. not an electrode), and an associated electrode 2600 can be positioned adjacent (e.g. attached to) and/or at least proximate the stimulation element 260 (as shown in FIG. 1). For example, one or more stimulation elements 260 can comprise one or more therapy delivery elements, such as: a light energy delivering component (e.g. a lens or a prism configured to deliver laser or other light energy), a sound energy delivering component (e.g. a piezo transducer configured to deliver ultrasound or other sound energy), and/or an agent delivering component (e.g. a needle or an outlet of a lumen), such as when each stimulation element 260 is attached to lead 265 with an electrode 2600 positioned adjacent to (e.g. attached to) and/or at least in close proximity to the stimulation element 260. In some embodiments, one or more stimulation elements 260 can comprise a sensor (e.g. all or a portion of stimulation elements 260 comprise a sensor), and an associated electrode 2600 can be positioned adjacent (e.g. attached to) and/or at least proximate the stimulation element 260, such as when delivery device 200 is configured as a diagnostic device to measure one or more physiologic parameters of a patient.

Apparatus 10 can include one or more devices for transferring power (e.g. via a wired or wireless connection) to delivery device 200, such as external device 500 shown. Alternatively or additionally, external device 500 can be configured to transfer data to, and/or receive data from, delivery device 200 (e.g. via a wired or wireless connection). In some embodiments, external device 500 is configured to be positioned (e.g. via a temporary adhesive and/or strap) above a location in which delivery device 200 has been implanted, and to at least wirelessly transfer power to the implanted delivery device 200.

Apparatus 10 can include a device for programming, delivering data to, and/or otherwise controlling delivery device 200, such as programmer 600 shown (e.g. via control signals sent via a wired or wireless connection).

Apparatus 10 can include one or more devices for gathering information related to the patient and/or the environment of the patient, such as diagnostic assembly 62 shown. Diagnostic assembly 62 can comprise an assembly that is integrated (in whole or in part) into delivery device 200, external device 500, and/or programmer 600.

One or more components of apparatus 10 of FIG. 1 can have similar construction and arrangement to the similar components of apparatus 10 of FIG. 1A described herein. Alternatively or additionally, one or more components of apparatus 10 of FIG. 1 can have similar construction and arrangement to the similar components of the stimulation apparatus described in applicant's co-pending International PCT Patent Application Serial Number PCT/US2020/054150, titled “Stimulation Apparatus”, filed Oct. 2, 2020 [Docket Nos. 47476-719.601; NAL-025-PCT].

Referring now to FIG. 1A, a schematic anatomical view of an apparatus for providing a therapy to a patient is illustrated, consistent with the present inventive concepts. Apparatus 10 of FIG. 1A comprises implantable system 20 and external system 50. In some embodiments, apparatus 10 of FIG. 1A, and/or its components, are of similar construction and arrangement to those described in applicant's co-pending International PCT Patent Application Serial Number PCT/US2020/054150, titled “Stimulation Apparatus”, filed Oct. 2, 2020 [Docket Nos. 47476-719.601; NAL-025-PCT].

External system 50 transmits transmission signals to one or more components of implantable system 20. These transmission signals can comprise power and/or data. Implantable system 20 comprises one or more devices for delivering a therapy, delivery device 200, shown implanted beneath the skin of patient P. In some embodiments, implantable system 20 comprises multiple similar or dissimilar delivery devices 200 (singly or collectively delivery device 200). Each delivery device 200 can be configured to receive power and data from a transmission signal transmitted by external system 50, such as when stimulation energy delivered to the patient (e.g. to nerve or other tissue of the patient) by delivery device 200 is provided via wireless transmissions signals from external system 50. In some embodiments, implantable system 20 comprises at least two delivery devices, such as delivery device 200 and delivery device 200′ shown in FIG. 1A. Delivery device 200′ can be of similar construction and arrangement to delivery device 200, and/or it can include components of a different configuration. Each delivery device 200 comprises one or more housings, housing 210 shown, which surrounds various other components of device 200 (e.g. a power supply, a receiver, a controller, and/or an antenna, each as described herein). In some embodiments, one or more inner or outer surfaces (or portions of surfaces) of housing 210 includes an insulating and/or shielding layer (e.g. a conductive electromagnetic shielding layer), such as inner coating 219a and/or outer coating 219b shown (singly or collectively coating 219). Coating 219 can comprise an electrically insulating and/or a thermally insulating layer or other coating. In some embodiments, one or more portions of housing 210 comprise an electrically shielding coating, coating 219, while other portions are transmissive to electromagnetic signals such as radiofrequency signals.

Each delivery device 200 comprises one or more stimulation and/or other functional elements, such as stimulation element 260 shown, where stimulation elements 260 are configured to deliver stimulation energy, a stimulating drug or other agent, and/or another form of stimulation (e.g. another form of tissue stimulation) to the patient. Alternatively or additionally, one or more stimulation elements 260 are configured as a sensor (e.g. when comprising an electrode configured to both deliver electrical energy and record electrical signals). Each delivery device 200 can include one or more leads, lead 265 shown, and each lead 265 can include one or more stimulation elements 260. Alternatively or additionally, one or more stimulation elements 260 can be positioned on housing 210 or one or more other components of delivery device 200. In some embodiments, delivery device 200 comprises at least two leads 265, such as is shown in FIG. 1.

Apparatus 10 can comprise one or more algorithms, algorithm 15 shown. Algorithm 15 can comprise one or more algorithms configured to determine location information regarding one or more leads 265, as described herein. In some embodiments, algorithm 15 comprises an algorithm that is based on data that is gathered prior to implantation of delivery device 200, such as data gathered during manufacturing of delivery device 200. For example, algorithm 15 can be based on electrode 2600 impedance data that was recorded prior to implantation of lead 265, such as when electrode 2600 comprises electrodes with a coating and/or an enhanced surface (e.g. resulting in a lowered impedance).

Each lead 265 can comprise one or more electrodes 2600 (four shown for lead 265 in FIG. 1A). In some embodiments, a single lead 265 comprises a set of at least 1, 2, 3, 4, 6, and/or 8 electrodes 2600. Each electrode 2600 can comprise a component configured to deliver current (also referred to as “source current” herein) and/or receive current (also referred to as “sink current” herein). In some embodiments, housing 210 comprises at least a portion that is conductive and configured as an electrode (e.g. configured to source and/or sink current as described herein). Current transmitted between two or more electrodes 2600 (via tissue in between the two or more electrodes 2600) can be used by apparatus 10 (e.g. used by algorithm 15) to identify (e.g. provide information related to) one or more of the following: the current position of one or more leads 265; the change in position of one or more leads 265; the relative position between two or more leads 265; the change in the relative position between two or more leads 265; and combinations of these, such as is described herein. In some embodiments, algorithm 15 is configured to provide lead 265 location information as described in reference to FIGS. 2-6 herein. In some embodiments, one or more electrodes 2600 comprise the same component as an associated set of one or more stimulation elements 260, such as the four electrodes 2600 that comprise the same four elements 260 of FIG. 1A.

Each delivery device 200 can comprise one or more other types of functional elements, such as functional element 299a shown positioned proximate housing 210 (e.g. within and/or on the external surface of housing 210) and/or functional element 299b shown positioned on lead 265. Functional element 299a and/or 299b (singly or collectively functional element 299) can comprise a transducer, a sensor, and/or other functional element as described herein. In some embodiments, a functional element 299 comprises a visualizable marker, such as a radiopaque marker, an ultrasonically visible marker, and/or a magnetic marker.

External system 50 can comprise an external device 500, which includes one or more housings, housing 510 shown, which surrounds various other components of device 500. In some embodiments, external system 50 comprises multiple external devices 500 (singly or collectively external device 500), such as an external device as is described in applicant's co-pending U.S. patent application Ser. No. 16/104,829, titled “Apparatus with Enhanced Stimulation Waveforms”, filed Aug. 17, 2018 [Docket nos. 47476-708.301; NAL-014-US]. In some embodiments, external system 50 comprises at least two, or at least three external devices (e.g. at least two external devices configured to deliver power and/or data to one or more delivery devices 200), such as external device 500, external device 500′, and external device 500″ shown in FIG. 1A. External device 500′ and/or 500″ can be of similar construction and arrangement to external device 500, and these devices can include components of a different configuration.

External system 50 can comprise one or more programming devices, programmer 600, such as patient programmer 600′ and/or clinician programmer 600″ shown. Patient programmer 600′ and clinician programmer 600″ (singly or collectively programmer 600) each comprise a user interface, such as user interfaces 680′ and 680″ shown (singly or collectively user interface 680). Programmer 600 can be configured to control one or more external devices 500. Alternatively or additionally, programmer 600 can be configured to control one or more delivery devices 200 (e.g. when no external device 500 is included in apparatus 10 or at least no external device 500 is available to communicate with a delivery device 200). Patient programmer 600′ can be configured to be used by the patient, patient caregiver (e.g. clinician of the patient), and/or a family member of the patient. In some embodiments, one or more external devices 500 comprise all or a portion of a programmer 600, such as when all or a portion of user interface 680 is integrated into housing 510 of external device 500. In some embodiments, apparatus 10 comprises multiple programmers 600, such as one or more patient programmers 600′ and/or one or more clinician programmers 600″.

Clinician programmer 600″ can be of similar construction and arrangement to patient programmer 600′. In some embodiments, clinician programmer 600″ provides additional functions not available in patient programmer 600′. In some embodiments, clinician programmer 600″ can modify the programming of patient programmer 600′ (e.g. modify the programming options available to the patient or family member of the patient).

External system 50 can comprise one, two, three, or more functional elements, such as functional elements 599a, 599b, and/or 599c (singly or collectively functional element 599), shown positioned in external device 500, patient programmer 600′, and clinician programmer 600″, respectively. Each functional element 599 can comprise a functional element as defined hereabove (e.g. a sensor, a transducer, and/or other functional element as described herein). In some embodiments, a functional element 599 comprises a needle, a catheter (e.g. a distal portion of a catheter), an iontophoretic element or a porous membrane, such as an agent delivery element configured to deliver one or more agents contained (e.g. one or more agents in a reservoir, such as reservoir 525 shown) within an external device 500 and delivered into the patient (e.g. into subcutaneous tissue, into muscle tissue and/or into a blood vessel such as a vein).

As described hereabove, external system 50 can be configured to transmit power and/or data (e.g. implantable system 20 configuration data) to one or more delivery devices 200 of implantable system 20. Implantable system 20 configuration data provided by external system 50 (e.g. via one or more antennas, antenna 540 shown, of one or more external devices 500) can include when to initiate stimulation delivery (e.g. energy delivery), and/or when to stop stimulation delivery, and/or it can include data related to the value or change to a value of one or more stimulation variables as described hereabove. The configuration data can include a stimulation parameter such as an agent (e.g. a pharmaceutical agent) delivery stimulation parameter selected from the group consisting of: initiation of agent delivery; cessation of agent delivery; amount of agent to be delivered; volume of agent to be delivered; rate of agent delivery; duration of agent delivery; time of agent delivery initiation; and combinations of these. The configuration data can include a sensing parameter, such as a sensing parameter selected from the group consisting of: initiation of sensor recording; cessation of sensor recording; frequency of sensor recording; resolution of sensor recording; thresholds of sensor recording; sampling frequency of sensor recording; dynamic range of sensor recording; initiation of calibration of sensor recording; and combinations of these.

As described herein, external system 50 can comprise one or more external devices 500. External system 50 can comprise one or more antennas 540, such as when a single external device 500 comprises one or more antennas 540, and/or when multiple external devices 500 each comprise one or more antennas 540. The one or more antennas 540 can transmit power and/or data to one or more antennas, antennas 240, of implantable system 20, such as when a single delivery device 200 comprises one or more antennas 240, and/or when multiple delivery devices 200 each comprise one or more antennas 240.

In some embodiments, one or more external devices 500 are configured to transmit both power and data (e.g. simultaneously and/or sequentially) to one or more delivery devices 200. In some embodiments, one or more external devices 500 are further configured to receive data from one or more delivery devices 200 (e.g. via data transmitted by one or more antennas 240 of one or more delivery devices 200). Each external device 500 can comprise housing 510, a power supply 570, a transmitter 530, a controller 550, and/or one or more antennas 540, each shown in FIG. 1A and described herein. Each external device 500 can further comprise one or more functional elements 599a, such as a functional element comprising a sensor, electrode, energy delivery element, a magnetic-field generating transducer, and/or any transducer, also described in detail herebelow. In some embodiments, a functional element 599a comprises one or more sensors configured to monitor performance of external device 500 (e.g. to monitor voltage of power supply 570, quality of transmission of power and/or data to implantable system 20, temperature of a portion of an external device 500, and the like).

External system 50 can transmit power and/or data with a transmission signal comprising at least one wavelength, λ. External system 50 and/or implantable system 20 can be configured such that the distance between an external antenna 540 transmitting the power and/or data and one or more implantable antennas 240 receiving the power and/or data transmission signal is equal to between 0.1λ and 10.0λ, such as between 0.2λ and 2.0λ. In some embodiments, one or more transmission signals are delivered by a transmitter, transmitter 530, at a frequency range between 10 MHz and 10.6 GHz, such as between 0.1 GHz and 10.6 GHz, between 10 MHz and 3.0 GHz, between 40 MHz and 1.5 GHz, between 10 MHz and 100 MHz, between 0.902 GHz and 0.928 GHz, in a frequency range proximate to 40.68 MHz, in a frequency range proximate to 866 MHz, or approximately between 863 MHz and 870 MHz. Transmitter 530 can comprise a transmitter that produces a transmission signal with a power level between 0.01 W and 4.0 W, such as a transmission signal with a power level between 0.01 W and 2.0 W or between 0.2 W and 1.0 W.

Housing 510 can comprise an adhesive element, spacer 511 shown, which can be configured as an adhesive element that temporarily attaches an external device 500 to the patient's skin. Alternatively or additionally, housing 510 can be constructed and arranged to engage (e.g. fit in the pocket of) a patient attachment device, such as patient attachment device 70 described herein (e.g. a clip that is adhesively attached to the patient's skin).

In some embodiments, transmitter 530 (and/or another component of external system 50) is further configured as a receiver (e.g. can further include a receiver, in addition to a transmitter or include a transmitter that further functions as a receiver), such as to receive data from implantable system 20. For example, a transmitter 530 can be configured to receive data via one or more antennas 240 of one or more delivery devices 200. Data received can include patient information (e.g. patient physiologic information, patient environment information or other patient information) and/or information related to an implantable system 20 parameter (e.g. a delivery device 200 stimulation parameter and/or another configuration parameter as described herein).

Each power supply 570 (singly or collectively power supply 570) can be operably attached to a transmitter 530, and one or more other electronic components of each external device 500. Power supply 570 can comprise a power supplying and/or energy storage element selected from the group consisting of: battery; replaceable battery (e.g. via a battery door of housing 510); rechargeable battery; AC power converter; capacitor; and combinations of these. In some embodiments, power supply 570 is configured to provide a voltage of at least 3V. In some embodiments, power supply 570 is configured to provide a capacity between 1 Watt-hour and 75 Watt-hours, such as a battery or capacitor with a capacity of approximately 5 Watt-hours. In some embodiments, power supply 570 comprises an AC power source. Power supply 570 can include voltage and/or current control circuitry. Alternatively or additionally, power supply 570 can include charging circuitry, such as circuitry configured to interface a rechargeable battery with an external charging device.

Each external device 500 can include one or more user interface components, user interface 580 shown, such as to allow the patient or other user to adjust one or more parameters of apparatus 10. User interface 580 can include one or more user input components (e.g. buttons, slides, knobs, and the like) and/or one or more user output components (e.g. lights, displays and the like). In some embodiments, user interface 580 includes one or more controls configured to provide a water-ingress-resistant barrier.

In some embodiments, housing 210 comprises an array of feedthroughs, not shown. In some embodiments, housing 210 is surrounded (e.g. partially or fully surrounded) by a covering, such as a flexible and/or non-conductive covering, such as a covering made of an elastomer.

Each delivery device 200 can include one or more energy storage assemblies 270 (singly or collectively energy storage assembly 270). Each assembly 270 can comprise one or more implantable energy storage components, such as one or more batteries (e.g. rechargeable batteries) and/or capacitors (e.g. a supercapacitor). Energy storage assembly 270 can be configured to provide power to one or more of: one or more stimulation elements 260; controller 250; receiver 230; and combinations of these. In some embodiments, energy storage assembly 270 further provides power to one or more antennas 240 and/or circuitry configured to transmit data via antenna 240. In some embodiments, energy storage assembly 270 includes digital control for charge/discharge rates, voltage outputs, current outputs, and/or system power distribution and/or management.

Energy storage assembly 270 can comprise one or more capacitors with a single or collective capacitance between 0.01 μF and 10 F, such as a capacitance between 1 μF and 1.0 mF, or between 1 μF and 10 μF. The energy storage assembly 270 can comprise one or more capacitors with capacitance between 1 mF and 10 F, such as when energy storage assembly 270 comprises a super-capacitor and/or an ultra-capacitor. Such large capacitance can be used to store sufficient charge to maintain operation (e.g. maintain delivery of stimulation energy and/or delivery of an agent) without the use (e.g. sufficient proximity) of an associated external device 500. A capacitor or other energy storage element (e.g. a battery) can be chosen to provide sufficient energy to maintain operation for at least 30 seconds, at least 2 minutes, at least 5 minutes, at least 30 minutes, and up to several hours or more (e.g. during showering, swimming or other physical activity). In some embodiments, energy storage assembly 270 is configured to provide continuous and/or intermittent stimulation energy for at least one charge-balanced pulse (e.g. for the duration of at least one charge-balanced pulse). In some embodiments, a capacitor, battery or other energy storage element is configured to provide stimulation energy without receiving externally supplied power for periods of at least 1 hour, at least 1 day, at least 1 month or at least 1 year. Energy storage assembly 270 can comprise one or more capacitors with a breakdown voltage above 1.0V, such as a breakdown voltage above 1.5V, 4.0V, 10V, or 15V. In some embodiments, energy storage assembly 270 can comprise capacitors distributed outside of housing 210, such as when one or more capacitors are distributed along lead 265. Energy storage assembly 270 can comprise one or more capacitors with low self-leakage, such as to maintain stored energy for longer periods of time.

In some embodiments, during use (e.g. during a period of providing stimulation or other function) delivery device 200 receives power regularly from external system 50 (e.g. relatively continuously while delivery device 200 delivers stimulation energy), and energy storage assembly 270 comprises a relatively small battery or capacitor, such as a battery or capacitor that has an energy storage capacity of less than or equal to 0.6 Joules, 7 Joules or 40 Joules.

Delivery device 200 can include one or more controllers 250 (singly or collectively controller 250), which can be configured to control one or more stimulation elements 260, such as a stimulation element 260 comprising a stimulation-based transducer (e.g. an electrode or other energy delivery element) and/or a sensor (e.g. a physiologic sensor and/or a sensor configured to monitor a delivery device 200 parameter). In some embodiments, controller 250 is configured to transmit a stimulation signal (e.g. transmit stimulation energy configured in one or more stimulation waveforms) to one or more stimulation elements 260 (e.g. one or more stimulation elements 260 comprising an electrode and/or other energy delivery element), independent of the power signal received by one or more antennas 240 (e.g. independent of power transmitted by external system 50), such as by using energy stored in energy storage assembly 270. In these embodiments, the power signal and/or the RF path for the power signal can be adjusted to optimize power efficiency (e.g. by tuning matching network on transmitter 530 and/or receiver 230; configuring antennas 540 and/or 240 in an array; tuning operating frequency; duty cycling the power signal; adjusting antenna 540 and/or 240 position; and the like), and a stimulation signal can be precisely delivered (e.g. by using energy stored on energy storage assembly 270 and generating a stimulation signal locally on the delivery device 200) to ensure clinical efficacy. Also, if the power signal transmission (also referred to as “power link”) is perturbed unexpectedly, the stimulation signal can be configured so that it is not significantly affected (e.g. unaffected). In some configurations, the stimulation signal being delivered by one or more delivery devices 200 is insensitive to interference that may be present. In these embodiments, a power transmission signal and stimulation signal can vary in one or more of: amplitude; changes in amplitude; average amplitude; frequency; changes in frequency; average frequency; phase; changes in phase; average phase; waveform shape; pulse shape; duty cycle; polarity; and combinations of these.

Controller 250 can receive commands from a receiver, receiver 230, such as one or more commands related to one or more delivery device 200 configuration parameters selected from the group consisting of: stimulation parameter; data rate of receiver; data rate of data transmitted by the first delivery device 200 at least one implantable antenna 240; stimulation element 260 configuration; state of controller 250; antenna 240 impedance; clock frequency; sensor configuration; electrode configuration; power management parameter; energy storage assembly parameter; agent delivery parameter; sensor configuration parameter; and combinations of these.

Controller 250 and/or any other component of each delivery device 200 can comprise an integrated circuit comprising one or more components selected from the group consisting of: matching network; rectifier; DC-DC converter; regulator; bandgap reference; overvoltage protection; overcurrent protection; active charge balance circuit; analog to digital converter (ADC); digital to analog converter (DAC); current driver; voltage driver; digital controller; clock generator; data receiver; data demodulator; data modulator; data transmitter; electrode drivers; sensing interface analog front end; power management circuit; energy storage interface; memory register; timing circuit; and combinations of these.

One or more receivers 230 (singly or collectively receiver 230) can comprise one or more components, such as demodulator 231, rectifier 232, and/or power converter 233 shown in FIG. 1A. In some embodiments, receiver 230 can comprise a DC-DC converter such as a boost converter. Receiver 230 can comprise a data receiver, such as a data receiver including an envelope detector and demodulator and/or an envelope averaging circuit. In some embodiments, one or more antennas 240 separately connect to one or more receivers 230. In some embodiments, one or more antennas 240 connect to a single receiver 230, such as via a series connection or a parallel connection.

One or more delivery devices 200 can be configured to transmit a data signal to external system 50. In some embodiments, receiver 230 is configured to drive one or more antennas 240 to transmit data to external system 50 (e.g. receiver 230 is further configured as a transmitter that wirelessly transmits data to an antenna 540 of an external device 500). Alternatively or additionally, delivery device 200 can be configured to transmit a data signal by having receiver 230 adjust a load impedance to backscatter energy, such as a backscattering of energy which can be detected by external system 50. In some embodiments, data transmission is accomplished by receiver 230 manipulating a signal at a tissue interface, such as to transmit a data signal using body conduction.

Demodulator 231 can comprise circuitry that asynchronously recovers signals modulated on the power signal provided by external system 50, and that converts the modulated signals into digital signals. In some embodiments, demodulator 231 asynchronously recovers the modulated signal by comparing a dynamically generated moving average with the envelope, outputting a high voltage when the envelope is greater than the moving average and a low voltage when the envelope is less than the moving average. Data can then be extracted from this resulting digital signal from the width and/or amplitude of the pulses in the signal, according to the encoding method used by external system 50. In some embodiments, demodulator 231 recovers a digital signal that is used as timing information for a delivery device 200, similar to an on-chip clock. The recovered clock signal can also be used to synchronize an on-chip clock generator of controller 250, such as through the use of a frequency and/or phase locked loop (FLL or PLL).

Rectifier 232 can comprise a power signal rectifier, such as to provide power to the energy storage assembly 270 and/or controller 250. In some embodiments, rectifier 232 comprises one or more self-driven synchronous rectifier (SDSR) stages connected in charge-pump configuration, to boost the voltage from input RF amplitude to the rectifier to a higher voltage. The boosted voltage can directly charge energy storage assembly 270, or it can be further boosted by a DC-DC converter or boost converter. In some embodiments, rectifier 232 comprises diode-capacitor ladder stages instead of, or in addition to, SDSR stages. On-chip diodes, such as Schottky diodes, or off-chip diodes can be used in one or more rectifier 232 stages. For maximum efficiency, the rectification elements, such as diodes, can be optimized to minimize forward conduction and/or reverse conduction losses by properly sizing the components and selecting the appropriate number of stages based on the input RF voltage and load current.

Power converter 233 can comprise one or more voltage conversion elements such as DC-DC converters that boost or otherwise change the voltage to a desired level. In some embodiments, voltage conversion is achieved with a buck-boost converter, a boost converter, a switched capacitor, and/or charge pumps. One or more power converters 233 can interface with energy storage assembly 270 and charge up associated energy storage components to desired voltages. In some embodiments, power converter 233 receives control signals from controller 250, such as to configure voltages, currents, charge/discharge rates, switching frequencies, and/or other operating parameters of power converter 233.

In some embodiments, delivery device 200 comprises one or more antennas 240 positioned on a substrate, such as a printed circuit board (PCB), a flexible printed circuit board and/or a foldable substrate (e.g. a substrate comprising rigid portions and hinged portions). In some embodiments, the substrate is folded or otherwise pivoted to position the various antennas 240 on differently oriented planes, such as multiple planes oriented between 5° and 90° relative to each other, such as two antennas 240 positioned on two planes oriented between 30° and 90° or between 40° and 90° relative to each other, or three antennas 240 positioned on three planes oriented between 5° and 60° relative to each other. Two or more antennas 240 can be positioned on two or more different planes that are approximately 45° relative to each other, or approximately 60° or approximately 90° relative to each other.

One or more antennas 240 can comprise an antenna selected from the group consisting of: loop antenna; multiple-turn loop antenna; planar loop antenna; coil antenna; dipole antenna; electric dipole antenna; magnetic dipole antenna; patch antenna; loaded dipole antenna; concentric loop antenna; loop antenna with ferrite core; and combinations of these. One or more antennas 240 can comprise a loop antenna, such as an elongated loop antenna or a multiple-turn loop antenna.

One or more antennas 240 can comprise a minor axis and a major axis. In some embodiments, one or more antennas 240 comprise a minor axis between 1 mm and 8 mm, such as between 2 mm and 5 mm. In some embodiments, one or more antennas 240 comprise a major axis between 3 mm and 15 mm, such as between 4 mm and 8 mm. In some embodiments, one or more antennas 240 comprise a major axis above 3 mm, such as between 3 mm and 15 mm, such as when the antenna 240 is positioned outside of housing 210.

One or more antennas 240 can be positioned inside of housing 210. Alternatively or additionally, one or more antennas 240 can be positioned outside of housing 210. Implantable system 20, one or more delivery devices 200 and/or one or more antennas 240 can be configured to be positioned at a desired depth beneath the patient's skin, such as at a depth between 0.5 cm and 7.0 cm, such as a depth of between 1.0 cm and 3.0 cm.

One or more implantable leads 265 (singly or collectively lead 265) can be attached to one or more housings 210, such as a lead 265 comprising one or more stimulation elements 260. Lead 265 can comprise one or more stimulation elements 260 configured as a stimulation element (e.g. an electrode configured to deliver electrical energy in monopolar or bipolar mode or an agent delivery element such as an output port fluidly connected to a reservoir within housing 210). Alternatively or additionally, lead 265 can comprise one or more stimulation elements 260 and/or functional elements 299b that is configured as a physiologic sensor (e.g. an electrode configured to record electrical activity of tissue or another physiologic sensor as described herein). Alternatively or additionally, lead 265 can comprise one or more stimulation elements 260 and/or functional elements 299b that is configured to transmit signals through tissue to external system 50, such as through body conduction.

In some embodiments, delivery device 200 comprises a connector, connector 215, that operably attaches (e.g. electrically attaches) one or more stimulation elements 260 to one or more components (e.g. electronic components) internal to housing 210 (e.g. to transfer power and/or data therebetween). In some embodiments, connector 215 is operably attached (e.g. in a manufacturing process) or attachable (e.g. in a clinical procedure) to lead 265 as shown in FIG. 1A. Alternatively, connector 215 can be operably attached and/or attachable to a lead connection assembly, assembly 280, which in turn can be attached to a lead 265. In some embodiments, connector 215 passes through an opening in housing 210, in a feed-through arrangement. In some embodiments, an overmold or other sealing element, sealing element 205 shown, provides a seal about connector 215, the opening in housing 210 and/or the interface between connector 215 and housing 210.

In some embodiments, lead 265 comprises a diameter between 1 mm and 4 mm, such as a diameter between 1 mm and 2 mm, such as a lead with a diameter of approximately 1.35 mm. In some embodiments, lead 265 comprises a length between 3 cm and 60 cm, such as a length between 6 cm and 30 cm. One or more leads 265 can include between 2 and 64 stimulation elements 260, such as when a lead 265 comprises between 2 and 64 electrodes, such as between 4 and 32 electrodes. In some embodiments, lead 265 comprises a paddle lead. In some embodiments, lead 265 comprises a single or multi-lumen catheter, such as when an attached delivery device 200 is configured as an agent delivery apparatus as described herein (e.g. a stimulation element 260 configured as a catheter comprises at least a portion of lead 265).

In some embodiments, lead 265 comprises one or more tines, such as tines 266 shown. Tines 266 can be configured to anchor or otherwise stabilize (“anchor” or “stabilize” herein) lead 265 relative to patient tissue, such as to prevent undesired movement during and/or after an implantation procedure for lead 265. One or more tines 266 can be configured to biodegrade after implantation in the patient, such that the stabilization provided is temporary. Tines 266 can be configured to biodegrade over a time period of approximately 4 to 12 weeks. In some embodiments, biodegradable tines 266 are configured to be incorporated when lead 265 stimulation elements 260 are positioned to stimulate a peripheral nerve (e.g. lead 265 is implanted such that one or more stimulation elements 260 are positioned proximate one or more peripheral nerves).

As described herein, one or more leads 265 can be positioned to stimulate the spinal cord, such as via percutaneous insertion of a lead 265 in the epidural space or surgical implantation of the lead 265 (e.g. a paddle lead) in the epidural space. A lead 265 can be placed such that one or more stimulation elements 260 (e.g. one or more electrodes) are positioned from T5-S5, such as to capture the area of pain or reduced circulation of the leg or foot. One or more stimulation elements 260 of one or more leads 265 can be positioned from C2 to T8, such as to capture the area of pain or reduced circulation of the arm or hand. One or more leads 265 can be placed along the midline, unilaterally and/or bilaterally over the dorsal columns, in the gutter (over dorsal roots) and/or in the dorsal root entry zone. Leads 265 can span several vertebral levels or they can be positioned to span a single level.

One or more stimulation elements 260 (singly or collectively stimulation element 260) and/or functional element 299 (e.g. functional element 299a and/or 299b) can comprise one or more sensors, transducers and/or other functional elements. In some embodiments, one or more stimulation elements 260 and/or functional elements 299 comprise at least one sensor and/or at least one transducer (e.g. a single stimulation element 260 or multiple stimulation elements 260). In some embodiments, stimulation element 260 and/or functional element 299 comprises a functional element configured to provide a therapy, such as one or more stimulation elements 260 configured to deliver an agent to tissue (e.g. a needle or catheter), to deliver energy to tissue and/or to otherwise therapeutically affect tissue. In some embodiments, stimulation element 260 and/or functional element 299 comprises one or more functional elements configured to record patient information, such as when stimulation element 260 and/or functional element 299 comprises one or more sensors configured to measure a patient physiologic parameter, as described herein. In some embodiments, stimulation element 260 and/or functional element 299 comprises one or more sensors configured to record a delivery device 200 parameter, also as described herein.

One or more stimulation elements 260 can be positioned on lead 265 as shown in FIG. 1A. Alternatively or additionally, one or more stimulation elements 260 can be positioned on housing 210. One or more functional elements 299 can be positioned on lead 265 (e.g. functional element 299b shown) and/or positioned on and/or within housing 210 (e.g. functional element 299a shown).

Stimulation element 260 can comprise one or more stimulation elements positioned at one or more internal body locations. Stimulation element 260 can comprise one or more stimulation elements positioned to interface with (e.g. deliver energy to and/or record a physiologic parameter from) spinal cord tissue, spinal canal tissue, epidural space tissue, spinal root tissue (dorsal or ventral), dorsal root ganglion, nerve tissue (e.g. peripheral nerve tissue, spinal nerve tissue or cranial nerve tissue), brain tissue, ganglia (e.g. sympathetic or parasympathetic) and/or a plexus. In some embodiments, stimulation element 260 comprises one or more elements positioned proximate and/or within one or more tissue types and/or locations selected from the group consisting of: one or more nerves; one or more locations along, in and/or proximate to the spinal cord; peripheral nerves of the spinal cord including locations around the back; the knee; the tibial nerve (and/or sensory fibers that lead to the tibial nerve); the occipital nerve; the sphenopalatine ganglion; the sacral and/or pudendal nerve; brain tissue, such as the thalamus; baroreceptors in a blood vessel wall, such as in the carotid artery; one or more muscles; the medial nerve; the hypoglossal nerve and/or one or more muscles of the tongue; cardiac tissue; the anal sphincter; the dorsal root ganglion; motor nerves; muscle tissue; the spine; the vagus nerve; the renal nerve; an organ; the heart; the liver; the kidney; an artery; a vein; bone; and combinations of these, such as to stimulate and/or record data from the tissue and/or location in which the stimulation element 260 is positioned proximate to and/or within. In some embodiments, apparatus 10, delivery device 200 and/or stimulation element 260 are configured to stimulate spinal nerves, peripheral nerves and/or other tissue as described in applicant's co-pending U.S. patent application Ser. No. 16/993,999, titled “Apparatus for Peripheral or Spinal Stimulation”, filed Aug. 14, 2020 [Docket nos. 47476-707.302; NAL-012-US-CON1].

In some embodiments, stimulation element 260 and/or functional element 299 comprises one or more sensors configured to record data representing a parameter of delivery device 200. In these embodiments, stimulation element 260 and/or functional element 299 can comprise one or more sensors selected from the group consisting of: an energy sensor; a voltage sensor; a current sensor; a temperature sensor (e.g. to record a temperature of one or more components of delivery device 200); a contamination detector (e.g. to detect undesired material that has passed through housing 210); an antenna matching and/or mismatching assessment sensor; power transfer sensor; link gain sensor; power use sensor; energy level sensor; energy charge rate sensor; energy discharge rate sensor; impedance sensor; load impedance sensor; instantaneous power usage sensor; average power usage sensor; bit error rate sensor; signal integrity sensor; and combinations of these. Apparatus 10 can be configured to analyze (e.g. via implantable controller 250, programmer 600 and/or diagnostic assembly 62 described herein) the data recorded by stimulation element 260 and/or functional element 299 to assess one or more of: power transfer; link gain; power use; energy within energy storage assembly 270; performance of energy storage assembly 270; expected life of energy storage assembly 270; discharge rate of energy storage assembly 270; ripple or other variations of energy storage assembly 270; matching of antenna 240 and 540; communication error rate between delivery device 200 and external device 500; integrity of transmission between delivery device 200 and external device 500; and combinations of these. A stimulation element 260 can be configured to record temperature, such as when apparatus 10 is configured to deactivate or otherwise modify the performance of a delivery device 200 when the recorded temperature exceeds a threshold.

In some embodiments, one or more stimulation elements 260 comprise a transducer configured to deliver energy to tissue, such as to treat pain and/or to otherwise stimulate or affect tissue. In some embodiments, stimulation element 260 comprises a stimulation element, such as one or more transducers selected from the group consisting of: an electrode; an energy delivery element such as an electrical energy delivery element, a light energy delivery element, a laser light energy delivery element, a sound energy delivery element, a subsonic sound energy delivery element and/or an ultrasonic sound delivery element; an electromagnetic field generating element; a magnetic field generating element; a mechanical transducer (e.g. delivering mechanical energy to tissue); a tissue manipulating element; a heat generating element; a cooling (e.g. cryogenic or otherwise heat extracting energy) element; an agent delivery element such as a pharmaceutical drug delivery element; and combinations of these. In some embodiments, one or more stimulation elements 260 comprise one or more electrodes configured to deliver energy to tissue and/or to sense a patient parameter (e.g. electrical activity of tissue or other patient physiologic parameter). In these embodiments, one or more stimulation elements 260 can comprise one or more electrodes selected from the group consisting of: microelectrode; cuff electrode; array of electrodes; linear array of electrodes; circular array of electrodes; paddle-shaped array of electrodes; bifurcated electrodes; and combinations of these.

In some embodiments, one or more stimulation elements 260 comprises a drug or other agent delivery element, such as a needle, port, iontophoretic element, catheter, or other agent delivering element that is connected to a reservoir of agent positioned within housing 210 (e.g. reservoir 225 shown). In some embodiments, one or more stimulation elements 260 comprise a drug eluting element configured to improve biocompatibility of implantable system 20.

In some embodiments, apparatus 10 (e.g. via stimulation element 260, functional element 299, and/or functional element 599) is configured to both record one or more patient parameters, and also to perform a medical therapy (e.g. stimulation of tissue with energy and/or an agent). In these embodiments, the medical therapy can be performed in a closed-loop fashion, such as when energy and/or agent delivery is modified based on the measured one or more patient physiologic parameters.

One or more portions of delivery device 200 or other component of implantable system 20 can be configured to be visualized or contain a visualizable portion or other visualizable element, such as visualizable element 222 shown. Visualizable element 222 can comprise a material selected from the group consisting of: radiopaque material; ultrasonically reflective material; magnetic material; and combinations of these. In these embodiments, each delivery device 200 can be visualized (e.g. during and/or after implantation) via an imaging device such as a CT, X-ray, fluoroscope, ultrasound imager and/or MRI.

In some embodiments, delivery device 200 and/or another component of apparatus 10 can include one or more features to prevent or at least reduce migration of delivery device 200 within the patient's body. In some embodiments, one or more delivery devices 200 comprises one or more anchor elements configured to secure one or more portions of delivery device 200 to tissue, such as anchor element 223 shown. Anchor element 223 can comprise one or more anchoring elements selected from the group consisting of: a sleeve such as a silicone sleeve; suture tab; suture eyelet; bone anchor; wire loops; porous mesh; penetrable wing; penetrable tab; bone screw eyelet; tine; pincers; suture slits; and combinations of these. While anchor element 223 is shown proximate housing 210 (e.g. to fixedly attach housing 210 to tissue), in some embodiments anchor element 223 surrounds or is otherwise proximate lead 265 (e.g. to fixedly attach lead 265 to tissue). In some embodiments, anchor element 223 comprises a porous mesh that surrounds all or a portion of housing 210. The porous mesh can be configured to promote tissue ingrowth, such as to prevent or at least limit (“prevent” herein) migration of housing 210 when delivery device 200 is implanted in the patient. In some embodiments, anchor element 223 comprises a mesh that is attached to the top side of delivery device 200 (side in closest proximity to the patient's skin), such as to prevent housing 210 from migrating away from the patient's skin (e.g. prevent from migrating deeper into the patient).

In some embodiments, apparatus 10 comprises one or more tools, tool 60 shown. Tool 60 can comprise a data logging and/or analysis tool configured to receive data from external system 50 or implantable system 20, such as data comprising: diagnostic information recorded by external system 50 and/or implantable system 20; therapeutic information recorded by external system 50 and/or implantable system 20; patient information (e.g. patient physiologic information) recorded by implantable system 20; patient environment information recorded by implantable system 20; and combinations of these. Tool 60 can be configured to receive data from wired or wireless (e.g. Bluetooth) means. Tool 60 can comprise a tool selected from the group consisting of: a data logging and/or storage tool; a data analysis tool; a network such as a LAN or the Internet; a cell phone; and combinations of these.

Apparatus 10 can include a battery charging assembly, charger 61 shown, such as an assembly configured to recharge one or more power supplies 570 and/or other component of apparatus 10 comprising a rechargeable battery or capacitor.

Apparatus 10 can include one or more implantation tools, tool 65 shown. Implantation tool 65 can comprise an introducer, tunneller, and/or other implantation tool constructed and arranged to aid in the implantation of housing 210, implantable antenna 240, lead 265 and/or one or more stimulation elements 260. In some embodiments, tool 65 comprises a component configured to anchor delivery device 200 to tissue, such as a mesh or wrap that slides around at least a portion of delivery device 200 and is configured to engage tissue (e.g. via tissue ingrowth) or be engaged with tissue (e.g. via suture or clips).

Apparatus 10 can include one or more placement tools, positioning tool 67 shown, which can be configured to aid in the positioning and/or maintenance of one or more external devices 500 on the patient's skin (e.g. at a location proximate an implanted delivery device 200).

Apparatus 10 can include one or more component positioning devices, such as patient attachment device 70 shown in FIG. 1A, that is used to attach one or more components of external system 50 to a location on or at least proximate the patient. Patient attachment device 70 can comprise one or more elements configured to attach one or more external devices 500 and/or programmer 600 at one or more locations on or proximate the patient's skin, that are relatively close to one or more delivery devices 200 that have been implanted in the patient. Patient attachment device 70 can comprise a component selected from the group consisting of: belt; belt with pockets; belt with adhesive; adhesive; strap; strap with pockets; strap with adhesive shoulder strap; shoulder band; shirt; shirt with pockets; clothing; clothing with pockets; epidural electronics packaging; clip (e.g. a clip that can be adhesively attached to the patient's skin); bracelet; wrist band; wrist watch; anklet; ankle bracelet; knee strap; knee band; thigh strap; thigh band; necklace; hat; headband; collar; glasses; goggles; earpiece; behind-the-earpiece; and combinations of these.

Apparatus 10 can comprise a device configured to operate (e.g. temporarily operate) one or more delivery devices 200, such as trialing interface 80 shown in FIG. 1A. Trialing interface 80 can be configured to wirelessly deliver power to a delivery device 200, wirelessly deliver data to a delivery device 200, and/or wirelessly receive data from a delivery device 200.

In some embodiments, apparatus 10 comprises a diagnostic assembly, diagnostic assembly 62 shown in FIG. 1A. In some embodiments, programmer 600 and/or implantable controller 250 comprise all or a portion of diagnostic assembly 62. Diagnostic assembly 62 can be configured to assess, monitor, determine and/or otherwise analyze patient information and/or delivery device 200 information, such as when one or more stimulation elements 260, functional elements 299, and/or functional elements 599 are configured as a sensor configured to record patient information (e.g. patient physiologic information and/or patient environment information) and/or apparatus 10 information (e.g. delivery device 200 information) as described herein.

In some embodiments, one or more stimulation elements 260 comprise a stimulation element configured to deliver energy (e.g. one or more electrodes configured to deliver monopolar or bipolar electrical energy) to tissue, and controller 250 is configured to control the energy delivery, such as to control one or more stimulation parameters. Each of these stimulation parameters can be held relatively constant, and/or varied, such as a variation performed in a continuous or intermittent manner. In some embodiments, one or more stimulation parameters are varied in a random or pseudo-random (hereinafter “random”) manner, such as a variation performed by apparatus 10 using a probability distribution as described in applicant's co-pending U.S. patent application Ser. No. 16/104,829, titled “Apparatus with Enhanced Stimulation Waveforms”, filed Aug. 17, 2018 [Docket nos. 47476-708.301; NAL-014-US]. In some embodiments, stimulation (e.g. stimulation comprising high frequency and/or low frequency signal components) is varied randomly to eliminate or at least reduce synchrony of neuronal firing with the stimulation signal (e.g. to reduce paresthesia or other patient discomfort). In some embodiments, one or more stimulation elements 260 comprise a stimulation element configured to stimulate a target (e.g. nerve tissue such as spinal nerve tissue and/or peripheral nerve tissue). The amount of stimulation delivered to the target can be controlled by varying a parameter selected from the group consisting of: stimulation element 260 size and/or configuration (e.g. electrode size and/or configuration); stimulation element 260 shape (e.g. electrode shape, magnetic field generating transducer shape or agent delivering element shape); shape of a generated electric field; shape of a generated magnetic field; stimulation signal parameters; and combinations of these.

In some embodiments, controller 250 is configured to produce a stimulation signal comprising a waveform or a waveform pattern (hereinafter stimulation waveform), for one or more stimulation elements 260 configured as an energy delivering stimulation element (e.g. such that one or more stimulation elements 260 deliver stimulation energy comprising or at least resembling that stimulation waveform). Controller 250 can produce a stimulation signal comprising a waveform selected from the group consisting of: square wave; rectangle wave; sine wave; sawtooth; triangle wave (e.g. symmetric or asymmetric); trapezoidal; ramp; waveform with exponential increase; waveform with exponential decrease; pulse shape which minimizes power consumption; Gaussian pulse shape; pulse train; root-raised cosine; bipolar pulses; and combinations of these. In some embodiments, controller 250 is configured to produce a stimulation signal comprising a waveform including a combination of two or more waveforms selected from the group consisting of: square wave; rectangle wave; sine wave; triangle wave (symmetric or asymmetric); ramp; waveform with exponential increase; waveform with exponential decrease; pulse shape which minimizes power consumption; Gaussian pulse shape; pulse train; root-raised cosine; bipolar pulses; and combinations of these. In some embodiments, controller 250 is configured to construct a custom waveform (e.g. an operator customized waveform), such as by adjusting amplitude at specified time steps (e.g. for one or more pulses). In some embodiments, controller 250 is configured to generate a waveform including one or more random parameters (e.g. random timing of pulses or random changes in frequency, rate of change or amplitude).

In some embodiments, controller 250 is configured to provide a stimulation signal comprising waveforms and/or pulses repeated at a frequency (e.g. includes a frequency component) between 1.0 Hz and 50 KHz, such as between 10 Hz and 500 Hz, between 40 Hz and 160 Hz and/or between 5 KHz and 15 KHz. In some embodiments, controller 250 is configured to produce a stimulation signal comprising a frequency between 1 Hz and 1000 Hz, such as a stimulation signal with a frequency between 10 Hz and 500 Hz. In some embodiments, controller 250 is configured to produce a stimulation signal comprising a duty cycle between 0.1% and 99%, such as a duty cycle between 1% and 10% or between 1% and 25%. In some embodiments, controller 250 is configured to produce a stimulation signal comprising a frequency modulated stimulation waveform, such as a stimulation waveform comprising a frequency component (e.g. signal) between 1 kHz and 20 kHz. In some embodiments, controller 250 is configured to produce a stimulation signal comprising a mix and/or modulation of low frequency and high frequency signals, which comprise any of the waveform types, shapes and other configurations. In these embodiments, the stimulation signal can comprise low frequency signals between 1 Hz and 1000 Hz, and high frequency signals between 600 Hz and 50 kHz, or between 1 kHz and 20 kHz. Alternatively or additionally, the stimulation signal can comprise a train of high frequency signals and bursts of low frequency signals, and/or a train of low frequency signals and bursts of high frequency signals. Alternatively or additionally, the stimulation signal can comprise one or more high frequency signals modulated with one or more low frequency signals, such as one or more high frequency signals frequency modulated (FM), amplitude modulated (AM), phase modulated (PM) and/or pulse width modulated (PWM) with one or more low frequency signals. The stimulation signal can cycle among different waveforms shapes at specified time intervals. The stimulation signal can comprise a pseudo random binary sequence (PRBS) non-return-to-zero or return-to-zero waveform, such as with a fixed and/or time-varying pulse width and/or frequency of the pulses.

In some embodiments, implantable system 20 of apparatus 10 is configured to provide paresthesia-reduced (e.g. paresthesia-free) high frequency pain management and rehabilitation therapy (e.g. via delivery of a stimulation signal above 600 Hz or 1 kHz, or other stimulation signal resulting in minimal paresthesia). Apparatus 10 can be configured to provide both low frequency (e.g. <1 kHz) stimulation and high frequency stimulation, such as when providing low frequency stimulation to elicit feedback from a patient during intraoperative or other (e.g. post-implantation) stimulation configuration. For example, trialing interface 80 can be used during an intra-operative titration of stimulation configuration using low frequency stimulation (e.g. to position and/or confirm position of one or more stimulation elements 260, such as to confirm sufficient proximity to target tissue to be stimulated and/or sufficient distance from non-target tissue not to be stimulated). In some embodiments, high frequency stimulation is delivered to reduce pain over extended periods of time, and low frequency stimulation is used in these intraoperative and/or post-implantation titration or other stimulation configuration procedures. Intentional elicitation of paresthesia (e.g. via low frequency stimulation and/or high frequency stimulation) is beneficial during stimulation element 260 (e.g. electrode) implantation because a patient can provide feedback to the implanting clinician to ensure that the stimulation elements 260 are positioned close to the target neuromodulation or energy delivery site. This implantation position-optimizing procedure can advantageously reduce the required stimulation energy due to stimulation elements 260 being closer to target tissue, since a minimum threshold for efficacious stimulation amplitude is proportional to the proximity of stimulation elements 260 to target tissue (e.g. target nerves). The patient can inform the clinician of the sensation of paresthesia coverage, and the clinician can adjust stimulation element 260 position to optimize stimulation element 260 location for efficacious treatment while minimizing unintentional stimulation of non-target tissue (e.g. motor nerves or other nerves which are not causing the patient's pain). These paresthesia-inducing techniques (e.g. using low frequency stimulation and/or high frequency stimulation) can be used during or after implantation of one or more delivery devices 200.

In some embodiments, apparatus 10 is configured to deliver low frequency stimulation energy (e.g. electrical energy comprising a low frequency signal) to stimulate motor nerves, such as to improve tone and structural support (e.g. physical therapy). In these embodiments, apparatus 10 can be further configured to provide high frequency stimulation, such as to treat pain (e.g. suppress and/or control pain). The combined effect can be used not only for pain management but also muscle strengthening and gradual healing of supportive structures. Alternatively or additionally, as described herein, apparatus 10 can be configured to deliver low frequency stimulation energy (e.g. electrical energy) to induce paresthesia, which can also be accompanied by the delivery of high frequency stimulation (e.g. to suppress and/or control pain). In some embodiments, apparatus 10 is configured to deliver low frequency stimulation (e.g. electrical energy comprising a low frequency signal) and burst stimulation, delivered simultaneously or sequentially. The low frequency stimulation and the burst stimulation can be delivered on similar and/or dissimilar stimulation elements 260 (e.g. similar or dissimilar electrode-based stimulation elements 260).

In some embodiments, implantable system 20 of apparatus 10 is configured to perform magnetic field modulation, such as targeted magnetic field neuromodulation (TMFN), electro-magnetic field neuromodulation, such as targeted electro-magnetic field neuromodulation (TEMFN), transcutaneous magnetic field stimulation (TMS), or any combination of these. Each delivery device 200, via one or more of its stimulation elements 260 (e.g. electrodes) can be configured to provide localized (e.g. targeted) magnetic and/or electrical stimulation. Combined electrical field stimulation and magnetic field stimulation can be applied by using superposition, and this combination can reduce the overall energy requirement. In some embodiments, implantable apparatus 10 comprises one or more stimulation elements 260 comprising a magnetic field generating transducer (e.g. microcoils or cuff electrodes positioned to partially surround or otherwise be proximate to one or more target nerves). Stimulation elements 260 comprising microcoils can be aligned with nerves to minimize affecting non-targeted tissue (e.g. to avoid one or more undesired effects to non-target tissue surrounding or otherwise proximate the target tissue). In some embodiments, the target tissue comprises dorsal root ganglia (DRG) tissue, and the non-target tissue comprises ventral root tissue (e.g. when the stimulation energy is below a threshold that would result in ventral root tissue stimulation).

One or more delivery devices 200 can be configured to deliver stimulation energy with a stimulation waveform that varies over time. In some embodiments, one or more stimulation parameters of the stimulation waveform are randomly varied over time, such as by using a probability distribution as described in applicant's co-pending U.S. patent application Ser. No. 16/104,829, titled “Apparatus with Enhanced Stimulation Waveforms”, filed Aug. 17, 2018 [Docket nos. 47476-708.301; NAL-014-US]. Each stimulation waveform can comprise one or more pulses, such as a group of pulses that are repeated at regular and/or irregular intervals. In some embodiments, a pulse can comprise delivery of electrical energy, such as electrical energy delivered in one or more phases (e.g. a pulse comprising at least a cathodic or anodic portion followed by passive capacitive recovery with an optional open circuit time between the first portion and recovery). In some embodiments, a group of pulses is delivered, each pulse comprising an anodic or cathodic portion that can include charge recovery after each pulse, such as charge recovery comprising active (opposite polarity pulse) recovery, and/or passive (capacitive) recovery. In some embodiments, there is no recovery between pulses, but instead active or passive recovery is included at the end of the set of the first (anodic or cathodic) portions. In some embodiments, single or groups of pulses are provided at time-varying modes of repetition (e.g. regular intervals for a period, then a period of irregular intervals) or at regular intervals with occasional (random) spurious pulses inserted (creating a single irregular event in an otherwise regular series). Non-limiting examples of waveform variations include: a variation in frequency (e.g. frequency of one or more signals of the waveform); variation of a signal amplitude; variation of interval time period (e.g. a time period between pulses or a time period between pulse trains); variation of a pulse width; multiple piecewise or continuous variations of one or more stimulation parameters in a single pulse (e.g. multi-step, multi-amplitude in one “super-pulse”); variation of pulse symmetry (e.g. via active drive, passive recovery and/or active-assisted passive recovery); variation of stimulation energy over a time window and/or overlapping time windows; variation of the power in the frequency spectrum of the stimulation waveform; and combinations of these. In some embodiments, apparatus 10 and/or delivery device 200 can be configured to vary a stimulation waveform “systematically” such as a variation performed temporally (e.g. on predetermined similar or dissimilar time intervals) and/or a variation performed based on a parameter, such as a measured parameter that can be based on a signal produced by a sensor of delivery device 200 or another component of apparatus 10. Alternatively or additionally, apparatus 10 and/or delivery device 200 can be configured to vary a stimulation waveform randomly. Random variation shall include discrete or continuous variations that can be selected from a distribution, such as a probability distribution selected from the group consisting of: a uniform distribution; an arbitrary distribution; a gamma distribution; a normal distribution; a log-normal distribution; a Pareto distribution; a Gaussian distribution; a Poisson distribution; a Rayleigh distribution; a triangular distribution; a statistic distribution; and combinations of these. Random pulses or groups of pulses can be generated based on randomly varying one or more stimulation signal parameters. One or more stimulation parameters can be varied randomly through the use of one or more probability distributions.

Apparatus 10 can be configured to stimulate tissue (e.g. stimulate nerve tissue such as tissue of the central nervous system or tissue of the peripheral nervous system, such as to neuromodulate nerve tissue), such as by having one or more delivery devices 200 deliver and/or otherwise provide energy (hereinafter “deliver energy”) and/or deliver an agent (e.g. a pharmaceutical compound or other agent) to one or more tissue locations, such as via one or more stimulation elements 260. In some embodiments, one or more delivery devices 200 deliver energy and/or an agent while receiving power and/or data from one or more external devices 500. In some embodiments, one or more delivery devices 200 deliver energy and/or an agent (e.g. continuously or intermittently) using energy provided by an internal power source (e.g. energy storage assembly 270) without receiving externally supplied power, such as for periods of at least 1 hour, at least 1 day, at least 1 month or at least 1 year. In some embodiments, one or more stimulation parameters are varied (e.g. systematically and/or randomly), during that period.

In some embodiments, apparatus 10 is further configured as a patient diagnostic apparatus, such as by having one or more delivery devices 200 record a patient parameter (e.g. a patient physiologic parameter) from one or more tissue locations, such as while receiving power and/or data from one or more external devices 500. In some embodiments, during its use, one or more delivery devices 200 receives at least power from one or more external devices 500 (e.g. with or without also receiving data). Alternatively or additionally, one or more patient parameters can be recorded by an external device of apparatus 10, such as via a programmer 600 and/or an external device 500.

Apparatus 10 can be configured as a patient information recording apparatus, such as by having one or more delivery devices 200 and/or one or more external devices 500 record patient information (e.g. patient physiologic information and/or patient environment information). In some embodiments, one or more delivery devices 200 and/or one or more external devices 500 further collect information (e.g. status information or configuration settings) of one or more of the components of apparatus 10.

In some embodiments, apparatus 10 is configured to deliver stimulation energy to tissue to treat pain. In particular, apparatus 10 can be configured to deliver stimulation energy to tissue of the spinal cord and/or tissue associated with the spinal cord (“tissue of the spinal cord”, “spinal cord tissue” or “spinal cord” herein), the tissue including roots, dorsal root, dorsal root ganglia, spinal nerves, ganglia, and/or other nerve tissue. The delivered energy can comprise energy selected from the group consisting of: electrical energy; magnetic energy; electromagnetic energy; light energy such as infrared light energy, visible light energy and/or ultraviolet light energy; mechanical energy; thermal energy such as heat energy and/or cryogenic energy; sound energy such as ultrasonic sound energy (e.g. high intensity focused ultrasound and/or low intensity focused ultrasound) and/or subsonic sound energy; chemical energy; and combinations of these. In some embodiments, apparatus 10 is configured to deliver to tissue energy in a form selected from the group consisting of: electrical energy such as by providing a controlled (e.g. constant or otherwise controlled) electrical current and/or voltage to tissue; magnetic energy (e.g. magnetic field energy) such as by applying controlled current or voltage to a coil or other magnetic field generating element positioned proximate tissue; and/or electromagnetic energy such as by providing both current to tissue and a magnetic field to tissue. A coil or other magnetic field generating element can surround (e.g. at least partially surround) the target nerve. Alternatively, or additionally, the magnetic energy can be applied externally and focused to specific target tissue via an implant comprising a coil and/or ferromagnetic materials. In some embodiments, the magnetic energy is configured to induce the application of mechanical energy. Delivered energy can be supplied in one or more stimulation waveforms, each waveform comprising one or more pulses of energy, as described in detail herebelow.

In some embodiments, apparatus 10 is configured as a stimulation apparatus in which external system 50 transmits a power signal to one or more delivery devices 200, and the one or more delivery devices 200 deliver stimulation energy to tissue with a stimulation signal (also referred to as a stimulation waveform), with the power signal and the stimulation signal having one or more different characteristics (e.g. as described herebelow). The power signal can be modulated with data (e.g. configuration or other data to be sent to one or more delivery devices 200). In these embodiments, the characteristics of the stimulation signal delivered (e.g. amplitude, frequency, duty cycle and/or pulse width), can be independent (e.g. partially or completely independent) of the characteristics of the power signal transmission (e.g. amplitude, frequency, phase, envelope, duty cycle and/or modulation). For example, the frequency and modulation of the power signal can change without affecting those or other parameters of the stimulation signal, and/or the parameters of the stimulation signal can be changed (e.g. via programmer 600), without requiring similar or any changes to the power signal. In some embodiments, implantable system 20 is configured to rectify the received power signal, and to produce a stimulation waveform with entirely different characteristics (e.g. amplitude, frequency and/or duty cycle) from the rectified power signal. Each delivery device 200 can comprise an oscillator and/or controller configured to produce the stimulation signal. In some embodiments, one or more delivery devices 200 is configured to perform frequency multiplication, in which multiple signals are multiplexed, mixed, added, and/or combined in other ways to produce a broadband stimulation signal.

In some embodiments, apparatus 10 is configured to treat a patient disease or disorder selected from the group consisting of: chronic pain; acute pain; migraine; cluster headaches; urge incontinence; pelvic dysfunction such as overactive bladder; fecal incontinence; bowel disorders; tremor; obsessive compulsive disorder; depression; epilepsy; inflammation; tinnitus; hypertension; heart failure; carpal tunnel syndrome; sleep apnea; obstructive sleep apnea; dystonia; interstitial cystitis; gastroparesis; obesity; mobility issues; arrhythmia; rheumatoid arthritis; dementia; Alzheimer's disease; eating disorder; addiction; traumatic brain injury; chronic angina; congestive heart failure; muscle atrophy; inadequate bone growth; post-laminectomy pain; liver disease; Crohn's disease; irritable bowel syndrome; erectile dysfunction; kidney disease; and combinations of these.

Apparatus 10 can be configured to treat heart disease, such as heart failure of a patient. In these embodiments, stimulation of the spinal cord can be performed. Apparatus 10 can be configured to pace and/or defibrillate the heart of a patient. One or more stimulation elements 260 can be positioned proximate cardiac tissue and deliver a stimulation signal as described herein (e.g. based on power and/or data received by implantable system 20 from external system 50). The stimulation signal can be used to pace, defibrillate and/or otherwise stimulate the heart. Alternatively or additionally, apparatus 10 can be configured to record cardiac activity (e.g. by recording EKG, blood oxygen, blood pressure, heart rate, ejection fraction, wedge pressure, cardiac output, lung impedance and/or other properties or functions of the cardiovascular system via a sensor-based element 260, and/or other sensor of apparatus 10), such as to determine an onset of cardiac activity dysfunction or other undesired cardiac state. In some embodiments, apparatus 10 is configured to both record cardiac or other information and deliver a stimulation signal to cardiac tissue (e.g. stimulation varied or otherwise based on the recorded information). For example, apparatus 10 can be configured such that external system 50 transmits power and/or data to implantable system 20. Implantable system 20 monitors cardiac activity, and upon detection of an undesired cardiovascular state, implantable system 20 delivers a pacing and/or defibrillation signal to the tissue that is adjacent to one or more stimulation elements 260 configured to deliver a cardiac stimulation signal.

As described hereabove, apparatus 10 can comprise an implantable system 20 which can include one or more delivery devices 200. Each delivery device 200 comprises a housing 210 and one or more leads 265, such as leads that are operator-attachable to housing 210 (e.g. attached in an implantation procedure), and/or fixedly attached to housing 210 (e.g. attached during a manufacturing process of delivery device 200).

Each lead 265 comprises one or more stimulation elements 260. Stimulation elements 260 can comprise electrical energy delivery elements (e.g. electrodes), electromagnetic energy delivery elements, light delivery elements, sound delivery elements, pharmaceutic drug and/or other agent delivery elements (e.g. needles and/or catheters), and/or other stimulation elements. Each lead 265 can be positioned (e.g. implanted by a clinician of the patient) to subsequently stimulate tissue (e.g. deliver stimulation energy and/or deliver a stimulating agent to stimulate tissue), such as when stimulation elements 260 are positioned in one or more anatomical locations to stimulate particular nerve tissue, such as to treat pain and/or provide another therapy to a patient. One or more stimulation elements 260 (e.g. positioned on one or more leads 265) can be positioned in the patient to perform spinal cord stimulation (SCS). Precise positioning of the stimulation elements 260 in the patient is related to the efficacy of the treatment (e.g. related to the amount of pain relief achieved).

Apparatus 10 can be constructed and arranged to prevent or at least reduce (“reduce” herein) migration of each lead 265 over time, where such migration can compromise the efficacy of stimulation energy delivery by the stimulation elements 260. Alternatively or additionally, apparatus 10 can be constructed and arranged to detect lead 265 migration, such as to detect magnitude of lead 265 migration and/or relative positioning changes of lead 265 after the migration.

Apparatus 10 can be configured to compensate for migration of one or more leads 265. For example, if a lead 265 migration is detected, one or more events can be performed, such as: an adjustment of stimulation energy delivery (e.g. pattern) delivered by the associated lead 265 (e.g. a stimulation pattern adjustment based on the detected migration); and/or a repositioning of lead 265 (e.g. in a surgical or other clinical procedure).

In some embodiments, each lead 265 can further comprise one or more electrodes 2600, such as electrodes that are used by algorithm 15 of apparatus 10 to assess migration of one or more leads 265 (e.g. amount of migration of one or more portions of a lead 265 from a first instance in time to a second instance in time) and/or identify the anatomical location of one or more leads 265 (e.g. identify the anatomical location of one or more portions of a lead 265 at the current instance in time). In some embodiments, one or more stimulation elements 260 of one or more leads 265 comprise an electrode that is configured to function as an electrode 2600 (e.g. a stimulation element 260 and the associated electrode 2600 are the same electrode). Alternatively or additionally, in some embodiments one or more stimulation elements 260 of one or more leads 265 are a non-electrode stimulation element, such as a non-electrode stimulation element selected from the group consisting of: a light delivery element (e.g. a lens or other optical component), a sound delivery element (e.g. an ultrasound delivery transducer); an electromagnetic energy delivery element; a pharmaceutical drug or agent delivery element (e.g. a needle, an opening in a catheter, and the like); and combinations thereof. In these embodiments, lead 265 can further comprise one or more electrodes 2600, such as one or more electrodes 2600 that are each positioned on lead 265 in close proximity to a non-electrode stimulation element 260, and/or located on another portion of lead 265. Whether electrodes 2600 are the same component as the stimulation elements 260, or whether stimulation elements 260 and electrodes 2600 comprise separate components positioned on a lead 265, electrodes 2600 can be used by algorithm 15 of apparatus 10 to determine the migration and/or anatomical location of stimulation elements 260 and/or one or more other portions of the associated lead 265, such as is described herein in reference to FIGS. 2-6.

Referring now to FIG. 2, a schematic view of two leads are illustrated, consistent with the present inventive concepts. Leads 265a and 265b each include multiple electrodes 2600 (eight elements shown for each lead in FIG. 2). In some embodiments, one or more of the electrodes 2600 are further configured as an electrical energy-delivering stimulation element 260. Alternatively or additionally, leads 265a and/or 265b include one or more non-electrical energy-delivering stimulation elements 260 (e.g. not shown, such as a stimulation element that is not an electrode, such as an element configured to deliver light energy, sound energy, and/or an agent), such as when one or more electrodes 2600 are each located in close proximity to an associated stimulation element 260, such as to provide migration and/or position information relative to the stimulation element 260 as described herein.

Referring additionally to FIGS. 3A-B, two implanted views of a pair of leads are illustrated, consistent with the present inventive concepts. In FIG. 3A, two leads 265a and 265b are shown in an originally implanted position. In FIG. 3B, a migration (e.g. of lead 265b and/or 265a) has occurred, such that leads 265a and/265b are positioned in a different position than that shown in FIG. 3A. Lx is a measure of the horizontal shift (as shown on the page) of lead 265b, and its associated stimulation elements 260b (electrodes 2600b shown), Ly is a measure of the vertical shift (as shown on the page) of lead 265b, and angle θ is a measure of angular rotation of lead 265b and its associated stimulation elements 260b (electrodes 2600b shown).

In some embodiments, apparatus 10 is configured to detect, quantify, and/or otherwise characterize (“characterize” herein) migration of one, two or more leads 265 (e.g. to characterize the migration between two or more leads 265), such as to use electrodes 2600 to characterize the migration of one or more stimulation elements 260.

As described herein, apparatus 10 can deliver electrical energy (i.e. current) to one or more electrodes 2600 (e.g. electrodes 2600 that are also stimulation elements 260 or otherwise), such as to perform multipolar stimulation or other current delivery between two or more electrodes (e.g. bipolar, tripolar, tetrapolar, and the like stimulation delivery). When current is delivered between multiple electrodes 2600, one or more is configured as a source of current, and one or more is configured as a sink of current. During current delivery, the voltage difference between the associated electrodes 2600 can be measured, and the impedance can be calculated by dividing this voltage difference by the amount of the supplied current (i.e. using Ohm's law). The measured voltage difference and/or the calculated impedance can be used (e.g. by algorithm 15) to characterize the migration, such as to estimate the horizontal shift Lx, the vertical shift Ly, and/or the angular rotation θ (representing the change in position between lead 265a and lead 265b).

As described hereabove, one or more stimulation elements 260 can comprise a stimulation element configured to deliver non-electrical stimulation energy, such as energy selected from the group consisting of: light energy such as laser light energy; sound energy; chemical energy such as is delivered by a pharmaceutical drug of other agent; magnetic energy; and combinations thereof. In these embodiments, all or some of the stimulation elements 260 can comprise an electrode-portion, electrode 2600, configured to deliver (i.e. source) and/or receive (i.e. sink) electrical current, such as in a bipolar, tripolar, tetrapolar, and/or other multipolar arrangement, such as to characterize migration of one or more leads 265.

In some embodiments, apparatus 10 comprises an algorithm, algorithm 15a, for characterizing migration of one or more leads 265. All or a portion of algorithm 15a can be stored in and/or utilized by delivery device 200, external device 500, and/or another component of apparatus 10. In these embodiments, apparatus 10 (e.g. the associated delivery device 200) can measure the impedance between two or more electrodes 2600 on a single lead 265 (e.g. adjacent electrodes 2600). Such as between electrodes 2600a1 and 2600a2 of lead 265a, or electrodes 2600b1 and 2600b2 of lead 265b. These impedance measurements can be taken for every pair of distinct electrodes 2600 on the same lead 265 (e.g. each adjacent pair and/or non-adjacent pair of electrodes 2600 on each lead 265). For each measurement, the associated separation distance between each two adjacent electrodes 2600 is known (e.g. as determined by the manufacture of each lead 265). Among the impedance measurements of adjacent electrodes 2600, there will be seven measurements for a lead 265 comprising eight electrodes 2600 as shown, correlating to 14 measurements collectively for lead 265a and 265b, with each of these impedance measurements correlating to an associated separation distance DS1 as shown. In some embodiments, each electrode 2600 is separated from an adjacent electrode 2600 by the same distance (electrodes 2600 are equidistantly spaced), such that the associated separation distances for all 14 measurements is the same distance. In these equidistant spacing embodiments, 12 pairs of electrodes 2600 on a single lead 265 will have a separation distance DS2 that is twice DS1, 10 pairs of electrodes 2600 on a single lead 265 will have a separation distance DS3 this is three times DS1, eight pairs of electrodes 2600 on a single lead 265 will have a separation distance DS4 this is four times DS1, six pairs of electrodes 2600 on a single lead 265 will have a separation distance DS5 this is five times DS1, four pairs of electrodes 2600 on a single lead 265 will have a separation distance DS6 this is six times DS1, and two pairs of electrodes 2600 on a single lead 265 will have a separation distance DS7 this is seven times DS1. Thus, a total of 56 inter-electrode 2600 impedance values can be measured on a single lead 265 (e.g. containing eight electrodes 2600). In some embodiments, two or more pairs of electrodes 2600 can be separated by dissimilar distances. Impedance measurements between any pair of electrodes 2600 (e.g. a majority of the pairs or all pairs of electrodes 2600) can be performed on each lead 265, and the associated separation distance D noted (e.g. by algorithm 15a).

Apparatus 10 (e.g. algorithm 15a) can fit a curve to the values of the impedances and associated distances, such as to obtain a function of impedance versus distance: Z=f(d) for a lead 265, where Z is the impedance calculated between the two electrodes 2600, and d is the associated distance between the two electrodes 2600. The function obtained is a continuous, non-linear function. The values of Z depend on the anatomy of the patient. In some embodiments, the values of Z vary from 500 ohms to 2,000 ohms. The values of d depend on electrode 2600 and lead 265 geometry, and their relative positions with respect to each other. In some embodiments, the distance d comprises a distance between 7 mm and 50 mm.

Apparatus 10 (e.g. delivery device 200) can be configured to measure the impedance between an electrode 2600 on a first lead (e.g. lead 265a shown) and an electrode 2600 on a second lead (e.g. lead 265b shown). In some embodiments, apparatus 10 can be configured to measure the impedance between all or a portion (e.g. a majority) of all combinations of pairs of electrodes 2600 existing (e.g. as implanted) between lead 265a and lead 265b. Every such pair of electrodes 2600 can be processed (e.g. impedance measurement between the two electrodes 2600 of the pair), such as when impedances for 64 electrode 2600 pairs is measured (e.g. correlating to eight electrodes 2600 on lead 265b for each electrode 2600 on lead 265a).

Algorithm 15a can estimate the distances between these electrode 2600 pairs, such as using the inverse function d=f⁻¹(Z). Each distance d can be calculated using the impedance value and the polynomial coefficients obtained while defining the function f. Thus, a total of 64 distance values can be obtained from the cross-lead 265 impedance measurements made from leads 265 (impedance measurements made from a pair of “cross-lead” electrodes comprising an electrode 2600 on a first lead 265 and an electrode 2600 on a second lead 265). As described herein, a configuration of Lx, Ly and θ defines relative position between leads 265a and 265b. This configuration is used to calculate the distance between each pair of electrodes 2600 between leads 265. The error between distances calculated for the configuration of Lx, Ly and θ and the distances obtained from the cross-lead 265 impedance measurements can be calculated. The sum of squared errors can be used as a closeness metric to evaluate the match between the two distance values. The values of Lx, Ly and θ that give the least sum of squared errors (best match) can be selected to represent the relative position of the leads 265.

In some embodiments, apparatus 10 includes (e.g. and utilizes) algorithm 15a, as described herein, when the implantation location of the associated leads 265 has uniform conductivity.

In some embodiments, apparatus 10 comprises an algorithm, algorithm 15b, for characterizing migration of one or more leads 265. All or a portion of algorithm 15b can be stored in and/or utilized by delivery device 200, external device 500, and/or another component of apparatus 10. In these embodiments, apparatus 10 (e.g. the associated delivery device 200) can measure the impedance between two or more electrodes 2600 on a single lead 265 (e.g. adjacent electrodes 2600 further configured as stimulation elements 260 or otherwise), such as between electrodes 2600a1 and 2600a2 of lead 265a, or electrodes 2600b1 and 2600b2 of lead 265b. These impedance measurements can be taken for every pair of distinct electrodes 2600 on the same lead 265 (e.g. each adjacent pair of electrodes 2600 on each lead 265). For each measurement, the associated separation distance between each two adjacent electrodes 2600 is known (e.g. as determined by the manufacture of each lead 265). Among the impedance measurements of adjacent electrodes 2600, there will be seven measurements for a lead 265 comprising eight electrodes 2600 as shown, correlating to 14 measurements collectively for lead 265a and 265b, with each of these impedance measurements correlating to an associated separation distance DS1 as shown. In some embodiments, each electrode 2600 is separated from an adjacent electrode 2600 by the same distance (electrodes 2600 are equidistantly spaced), such that the associated separation distances for all 14 measurements is the same distance. In these equidistant spacing embodiments, 12 pairs of electrodes 2600 on a single lead 265 will have a separation distance DS2 that is twice DS1, 10 pairs of electrodes 2600 on a single lead 265 will have a separation distance DS3 this is three times DS1, eight pairs of electrodes 2600 on a single lead 265 will have a separation distance DS4 this is four times DS1, six pairs of electrodes 2600 on a single lead 265 will have a separation distance DS5 this is five times DS1, four pairs of electrodes 2600 on a single lead 265 will have a separation distance DS6 this is six times DS1, and two pairs of electrodes 2600 on a single lead 265 will have a separation distance DS7 this is seven times DS1. Thus, a total of 56 inter-electrode 2600 impedance values can be measured on a single lead 265 (e.g. containing eight electrodes 2600). In some embodiments, two or more pairs of electrodes 2600 can be separated by dissimilar distances. Impedance measurements between any pair of electrodes 2600 (e.g. a majority of the pairs or all pairs of electrodes 2600) can be performed on each lead 265, and the associated separation distance D noted (e.g. by algorithm 15b).

Algorithm 15b can be configured to create a resistivity profile, which is estimated using the measurements of impedance and distance described hereabove. The resistivity profile is calculated based on resistivity values ρᵢⱼ, given by:

$${ρ}_{ij} = \frac{{Z}_{ij}}{{l}_{ij}}$$

where ρᵢⱼ, Zᵢⱼ, lᵢⱼ are the resistivity, impedance, and distance, respectively, between electrodes 2600i and 2600j of the same lead 265. After the resistivity values are calculated, algorithm 15b fits a polynomial spline curve to these values, to obtain a resistivity profile. The resistivity profile is a function of local resistivity versus distance from the first electrode (e.g. electrode 2600a1 or 2600b1), given by ρ=ƒ(d). The function ƒ is defined by the coefficients of the fitted polynomial. The function ƒ is a continuous, non-linear function. ρ is the local resistivity at a distance d from the first electrode 2600a1 for lead 265a (or 2600b1 for lead 265b). The function ƒ is obtained such that it fits the following mathematical model (“model” herein) to the measured ρᵢⱼ resistivity values.

ρᵢⱼ=∫_{di}^{dj}w(x)η(x)dx

where dᵢ indicates distance of i^{th} electrode from first electrode 2600a1 for lead 265a (or 2600b1 for lead 265b) and dᵢ<dⱼ. The weighing function w(x) is assumed to be known and ∫_{d}_{ᵢ}^{d}^{ⱼ}w(x)dx=1 and w(x)≥0. In some embodiments, the function w(x) is equal to

$$\frac{1}{\vert {d}_{i} - {d}_{j} \vert} .$$

in some embodiments, the function w(x) is symmetric about the point

$$\frac{\vert {d}_{i} + {d}_{j} \vert}{2}$$

and assigns larger weights to local resistivity values (φ near dᵢ and dⱼ, and smaller weights to local resistivity values in between dᵢ and dⱼ. One resistivity profile is estimated per lead 265. The values of ρ depend on the anatomy of the patient, and are expected to vary from 100 ohms/mm to 250 ohms/mm. In some embodiments, the distance d comprises a distance between 7 mm and 50 mm.

Estimation techniques based on impedance measurements are well known in the art. However, it is possible that electrodes 2600 remain in the same region but the distance between the electrodes 2600 on a lead 265 is different by design. It is also possible that the distance between the electrodes 2600 changes due to deformation of the lead 265 where the amount of deformation is known or measured. In these cases, the impedance measurements will change but the local resistivity values will remain the same. Therefore, in the estimation techniques used by algorithm 15b, resistivity value is used, as its value will remain the same, or change minimally, with changes in the distances between pairs of electrodes 2600.

Apparatus 10 (e.g. delivery device 200) can be configured to measure the impedance between an electrode 2600 on a first lead (e.g. lead 265a shown) and an electrode 2600 on a second lead (e.g. lead 265b shown). In some embodiments, apparatus 10 can be configured to measure the impedance between all or a portion (e.g. a majority) of all combinations of pairs of electrodes 2600 existing (e.g. as implanted) between lead 265a and lead 265b. Each such pair consists of one electrode 2600 from lead 265a and the other electrode 2600 from lead 265b. Every such pair of electrodes 2600 can be processed (e.g. impedance measurement between the two elements 260 of the pair), such as when impedances for 64 electrode 2600 pairs is measured (e.g. correlating to eight electrodes 2600 on lead 265b for each electrode 2600 on lead 265a).

Algorithm 15b can estimate the distances d between these pairs of electrodes 2600 across leads 265, such as using a linear resistivity assumption, such as given by:

$${l}_{ij} = \frac{{Z}_{ij}}{{ρ}_{ij}}$$

where, Zᵢⱼ is the measured impedance between elements 260i (on lead 265a) and 260j (on lead 265b) and

$${ρ}_{ij} = \frac{{ρ}_{i} + {ρ}_{j}}{2}$$

is the estimated resistivity and ρᵢ=ƒₐ(dᵢ), where ƒₐ is the resistivity profile for lead 265a and dᵢ is the distance of i^{th} electrode from 2600a1 and ρⱼ=ƒ_{b}(dⱼ) where ƒ_{b} is the resistivity profile for lead 265b and dᵢ is the distance of j^{th} electrode from 2600b1. Thus, a total of 64 distance values can be obtained from impedance measurements made from pairs of cross-lead 265 electrodes. As described herein, the values of Lx, Ly and θ are then selected such that the distances calculated in terms of Lx, Ly and θ closely match the distances obtained from the cross-lead 265 impedance measurements. Algorithm 15b uses the distance values (e.g. 64 distance values) to estimate the best fitting values of Lx, Ly, and θ.

In some embodiments, apparatus 10 includes (e.g. and utilizes) algorithm 15b, as described herein, when the implantation location of the associated leads 265 has uniform conductivity.

In some embodiments, apparatus 10 comprises an algorithm, algorithm 15c, for characterizing migration of one or more leads 265. All or a portion of algorithm 15c can be stored in and/or utilized by delivery device 200, external device 500, and/or another component of apparatus 10. In these embodiments, apparatus 10 (e.g. the associated delivery device 200) can measure the impedance between two or more electrodes 2600 on a single lead 265 (e.g. adjacent electrodes 2600), such as between electrodes 2600a1 and 2600a2 of lead 265a, or electrodes 2600b1 and 2600b2 of lead 265b. These impedance measurements can be taken for every pair of distinct electrodes 2600 on the same lead 265 (e.g. each adjacent pair of electrodes 2600 on each lead 265). For each measurement, the associated separation distance between each two adjacent electrodes 2600 is known (e.g. as determined by the manufacture of each lead 265). Among the impedance measurements of adjacent electrodes 2600, there will be seven measurements for a lead 265 comprising eight electrodes 2600 as shown, correlating to 14 measurements collectively for lead 265a and 265b, with each of these impedance measurements correlating to an associated separation distance DS1 as shown. In some embodiments, each electrode 2600 is separated from an adjacent electrode 2600 by the same distance (electrodes 2600 are equidistantly spaced), such that the associated separation distances for all 14 measurements is the same distance. In these equidistant spacing embodiments, 12 pairs of electrodes 2600 on a single lead 265 will have a separation distance DS2 that is twice DS1, 10 pairs of electrodes 2600 on a single lead 265 will have a separation distance DS3 this is three times DS1, eight pairs of electrodes 2600 on a single lead 265 will have a separation distance DS4 this is four times DS1, six pairs of electrodes 2600 on a single lead 265 will have a separation distance DS5 this is five times DS1, four pairs of electrodes 2600 on a single lead 265 will have a separation distance DS6 this is six times DS1, and two pairs of electrodes 2600 on a single lead 265 will have a separation distance DS7 this is seven times DS1. Thus, a total of 56 inter-electrode 2600 impedance values can be measured on a single lead 265 (e.g. containing eight electrodes 2600). In some embodiments, two or more pairs of electrodes 2600 can be separated by dissimilar distances. Impedance measurements between any pair of electrodes 2600 (e.g. a majority of the pairs or all pairs of electrodes 2600) can be performed on each lead 265, and the associated separation distance D noted (e.g. by algorithm 15c).

Algorithm 15c can be configured to create a resistivity profile, which is estimated using the measurements of impedance and distance described hereabove. The resistivity profile is calculated based on resistivity values ρij, given by:

$${ρ}_{ij} = \frac{{Z}_{ij}}{{l}_{ij}}$$

where ρᵢⱼ, Zᵢⱼ, lᵢⱼ are the resistivity, impedance, and distance, respectively, between electrodes 2600i and 2600j of the same lead 265. After the resistivity values are calculated, algorithm 15c fits a polynomial spline curve to these values, to obtain a resistivity profile. The resistivity profile is a function of local resistivity versus distance from the first electrode (e.g. electrode 2600a1 or electrode 2600b1), given by ρ=ƒ(d). The function ƒ is defined by the coefficients of the fitted polynomial. The function ƒ is a continuous, non-linear function. ρ is the local resistivity at a distance d from the first electrode 2600a1 for lead 265a (or 2600b1 for lead 265b). The function ƒ is obtained such that it fits the following model to the measured ρᵢⱼ resistivity values.

ρᵢⱼ=∫_{di}^{dj}w(x)ƒ(x)dx

where dᵢ indicates distance of i^{th} electrode from first electrode 2600a1 for lead 265a (or 2600b1 for lead 265b) and dᵢ<dⱼ. The weighing function w(x) is assumed to be known and ∫_{d}_{ᵢ}^{d}^{ⱼ}w(x)dx=1 and w(x)≥0. In some embodiments, the function w(x) is equal to

$$\frac{1}{\vert {d}_{i} - {d}_{j} \vert} .$$

In some embodiments, the function w(x) is symmetric about the point

$$\frac{\vert {d}_{i} + {d}_{j} \vert}{2}$$

and assigns larger weights to local resistivity values (φ near dᵢ and dⱼ, and smaller weights to local resistivity values in between dᵢ and dⱼ. The resistivity profile is a graph of local resistivity values versus distance from the first electrode. One such graph is estimated per lead 265. The values of p depend on the anatomy of the patient, and are expected to vary from 100 ohms/mm to 250 ohms/mm. The values of d depend on electrodes 2600 (e.g. the arrangement of electrodes 2600). In some embodiments, the distance d comprises a distance between 7 mm and 50 mm.

Algorithm 15c can calculate an “initial graph”, as described hereabove, during a patient “checkup” (e.g. a patient diagnostic procedure that occurs soon after implantation of the one or more leads 265, and/or at any time a position of one or more leads 265 is to be assessed or recorded). Subsequently, algorithm 15c can calculate one or more “subsequent graphs” (e.g. in a similar patient checkup procedure), such as to characterize lead 265 migration between two time periods (e.g. since implantation of leads 265). Each of these graphs represent local resistivity profiles observed at the time of measurement. Algorithm 15c can be configured to measure absolute and/or relative staggering between leads 265, such as vertical staggering between two or more leads 265, as described herebelow.

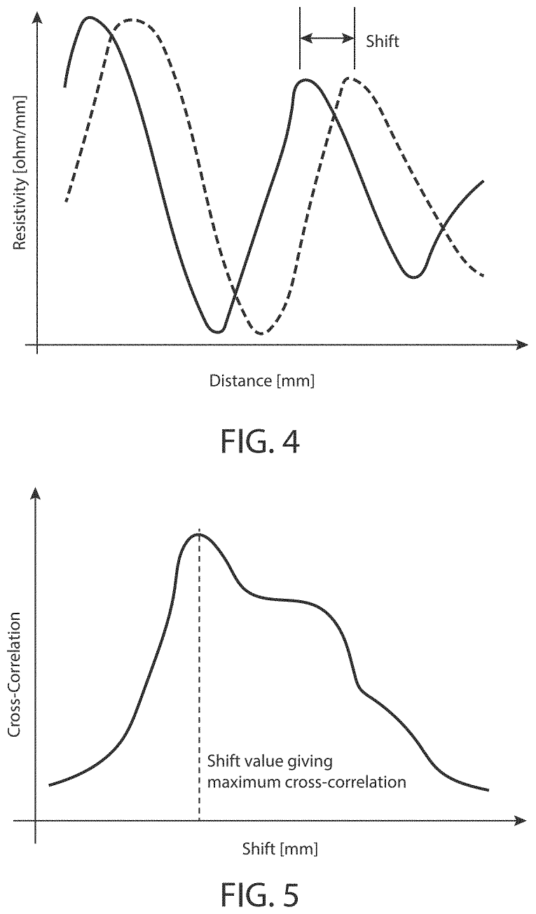

In some embodiments, an “absolute location estimation” is performed by algorithm 15c. Algorithm 15c can compare a subsequent graph with an initial graph, and a shift in the graphs can be calculated. Assuming that the resistivity profiles of the body do not change over time, the two graphs should be shifted versions of each other. The shift in the resistivity profile calculated from the graph by algorithm 15c corresponds to the vertical migration of a lead 265 with respect to the original position of that lead 265, as shown in FIG. 4. The dashed line curve in FIG. 4 represents the resistivity versus distance graph for the original lead 265 configuration. The solid line curve of FIG. 4 represents the resistivity versus distance graph for the migrated-lead 265 configuration. The shift in the graphs corresponds to the vertical migration of the lead.

In simulation experiments using algorithm 15c, performing cross-correlation between the initial graphs (resistivity profile) and subsequent graphs (resistivity profiles) corresponding to the migrated configuration was calculated (e.g. for each lead 265). The location of the maximum value of cross-correlation then provides an estimate of the amount of vertical migration of individual leads 265. FIG. 5 shows the variation in the value of cross-correlation for different values of shifts.

In some embodiments, a “relative location estimation” is performed by algorithm 15c, such as to estimate the relative vertical migration between two leads 265. Algorithm 15c can include one or more methods to estimate the relative migration. In a first method, the vertical migration of each lead 265 is estimated with respect to its original position, such as by calculating the cross-correlation as mentioned hereabove. Using the estimate of migration for each lead 265 with respect to its original position, algorithm 15c then calculates the relative migration between the two leads 265. Thus, in this first method, algorithm 15c calculates the relative positions of both leads 265 based on absolute position estimation. This method requires impedance measurements for the as-implanted lead 265 locations, as well as the impedance measurements for a subsequent, (potentially) migrated lead 265 location.

In a second method, algorithm 15c calculates the resistivity profile for both leads 265 using the impedance measurements. Algorithm 15c then calculates the cross-correlation between the resistivity profiles for the two leads 265. The location of the maximum value of cross-correlation provides an estimate of the relative migration between the two leads 265. In this second method, algorithm 15c assumes that the electrodes 2600 of two leads 265 that are close to each other have similar consecutive resistivity values (e.g. electrode resistivity values). Thus, using this second method, algorithm 15c can calculate the relative positions of the leads 265. This second method only requires the impedance measurements for the (potentially) migrated lead 265 locations (e.g. the as-implanted lead 265 locations are not needed).

In generating a resistivity profile, algorithm 15c can include impedance measurements from adjacent electrodes 2600 only, or more (e.g. all) electrode pairs from each electrode 2600 can be used. For example, 28 electrode 2600 pairs from each lead 265 can be used for the resistivity profile generation.

In some embodiments, the relative migration between two leads 265 is computed by algorithm 15c by finding the location of the maximum value of cross-correlation. Alternatively or additionally, the relative migration between the two leads can be computed by finding the relative migration location of the minimum value of the mean of square of differences (or absolute value of differences and the like) between resistivity profiles of the two leads 265.

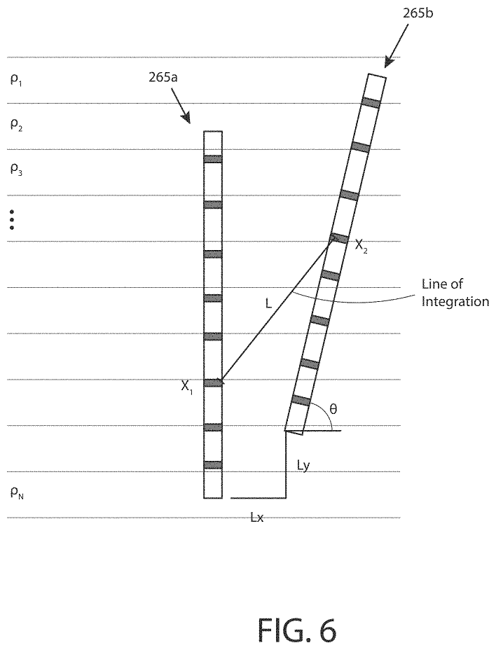

In some embodiments, apparatus 10 comprises an algorithm, algorithm 15d, for characterizing migration of one or more leads 265. All or a portion of algorithm 15d can be stored in and/or utilized by delivery device 200, external device 500, and/or another component of apparatus 10. In these embodiments, the patient's body is assumed to be made up of layers with constant resistivity, with the layers stacked on top of each other. A representative model of the layered construction of the patient's body is shown in FIG. 6. The impedance between two electrodes 2600 can be modeled as given in the below formula:

$${Z}_{ij} = {\int }_{0}^{L} ω ( l ) ρ ( {X}_{i} + ( {X}_{j} - {X}_{i} ) ( \frac{l}{L} ) ) dl + {Z}_{BIAS}$$

where, ω(l) is weighing function, ρ(X) resistivity at location X.

Zij represents impedance between i^{th} and i^{th} electrode 2600. In the formula, p is a function that gives the resistivity value at the given location and co is a weighing function. The above formula models impedance as a bias impedance Z_{BIAS} plus an integration of weighted resistivity values over a line joining two electrodes 2600 as shown in FIG. 6. Resistivity values can be negative as they model variations above the bias impedance Z_{BIAS}. Algorithm 15d can implement the above formula as a summation. The impedance between two electrodes 2600 is assumed to be the sum of the products of resistivity, weight and lengths in each layer between the two electrodes 2600. Based on an assumption that the patient's body has a layered construction, with each layer having a constant resistivity, the integration over a line can be written as a summation as follows:

$$Z = {\sum }_{k = i}^{j} {W}_{k} {ρ}_{k} {l}_{k} + {Z}_{BIAS}$$

where, ρₖwₖ and lₖ are the resistivity, weight, and length, respectively, of the kₜₕ segment.

In this model, Lx, Ly and θ, Z_{BIAS}, layer resistivities, and weight function are parameters of the model. Given these parameter values, impedance value between any two electrodes 2600 can be calculated by algorithm 15d based on the model.

Algorithm 15d can be used to determine relative location (Lx, Ly and θ) between the leads 265 or to determine the location of one or more leads 265 with respect to a previously known location of the leads 265 (e.g. relative to the original implant location, and/or a previously migrated to location). The impedance between all possible electrode 2600 pairs are used to determine the location. Selecting two electrodes 2600 from a total of 16 electrodes 2600 can be done in 120 (C2 16) different ways. Therefore, algorithm 15d uses 56 same-lead and 64 cross-lead impedance values.

In some embodiments, algorithm 15d is configured to perform a relative location estimation. Using certain layer lengths, Lx, Ly, θ and measured impedances, an equation is formulated for each impedance value as follows:

$$( Z = {\sum }_{k = i}^{j} {W}_{k} {ρ}_{k} {l}_{k} + {Z}_{BIAS} )$$

Solving this set of simultaneous equations gives the estimated resistivity for each layer, weights values and average impedance. As weight values are multiplied with resistivity values it is a system of non-linear set of equations. In some embodiments, a certain weighing function can be assumed, in which case the simultaneous equations can be solved as a linear system of equations. In some embodiments, the weighing function Wₖ is assumed to be known and ΣₖWₖ=land Wₖ≥0. In some embodiments, Wₖ is proportional to the length of the kth segment. In some embodiments, the function Wₖ is a constant. In some embodiments, the function Wₖ assigns larger weights to the resistivity values of segments at the extremities of the line (e.g. ρi, ρi+1, ρj−1, ρj) and smaller weights to the resistivity values of segments at the center of the line. The estimated resistivities are then used to obtain the estimated impedance values. The sum of squared differences between the measured impedance values and estimated impedance values can be calculated as an error metric between the match.

Algorithm 15d evaluates the error metric for different values of Lx, Ly and θ. The values for which the error is a minimum (e.g. is relatively minimized) are chosen as the relative location between two leads 265. In this configuration, the layers are aligned to one lead 265 and the relative location of the other lead 265 is estimated with respect to it. Applicant has conducted experiments in which it was observed that this configuration was able to give a resolution of 5 mm in estimation of stagger, providing a reliable method of estimating the relative positions of migrated leads 265. In this configuration, only impedance measurements are needed to perform the estimation.

In some embodiments, algorithm 15d is configured to perform an absolute location estimation. In these embodiments, the layer resistivities, weights, and other parameters estimated or used during a patient checkup are used (e.g. a diagnostic procedure that occurs soon after implantation of one or more leads 265), these particular set of parameters referred to as the “initial configuration”. Impedance values are measured for the migrated configuration of leads 265 for that patient. Given the parameters of the initial configuration and relative location (Lx, Ly and θ) of a migrated lead 265a with respect to lead 265a in the initial configuration, and relative location of migrated lead 265b with respect to lead 265a of the initial configuration, impedance between all pairs of electrodes 2600 are estimated using the model. The sum of square of differences between measured and estimated impedance values is used as a metric of the match. Algorithm 15d searches the relative location of migrated leads 265 for which the sum of square of difference is minimum and choses it as the new position of the migrated leads 265.

In some embodiments, algorithm 15 comprises one, two, three, or all of algorithms 15a, 15b, 15c, and/or 15d described herein. For example, algorithm 15 can comprise algorithm 15d used in combination with algorithm 15c, such as to provide more accurate lead 265 migration movement information and/or location information than the information that would be achieved if either algorithm was used alone. Algorithm 15d can be performed to provide an estimate of relative positions of leads 265, and algorithm 15c can be performed (e.g. also be performed) to provide an estimate of relative and absolute location of leads 265. Algorithm 15c provides an estimate (e.g. only provides an estimate) of vertical stagger of leads 265. Absolute vertical migration of leads 265 can be estimated using algorithm 15c, and these results can be used by algorithm 15d to estimate the relative location between two leads 265. Use of algorithms 15c and 15d can provide a more accurate estimate of absolute positions of multiple (e.g. two) leads 265.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

1.-57. (canceled)

58. A medical apparatus for a patient, comprising;
a delivery device comprising:
a plurality of electrodes comprising a first set of electrodes comprising one or more electrodes, and a second set of electrodes comprising one or more electrodes;
a first lead comprising the first set of electrodes; and
a second lead comprising the second set of electrodes,
wherein the delivery device is configured to measure impedance between multiple pairs of electrodes of the plurality of electrodes;
a processor operatively coupled to the delivery device; and
a memory operatively coupled to the processor and storing:
instructions for the processor to determine position information of the first lead and/or the second lead based on the measured impedances;
a mathematical model; and
a list of pairs of electrodes selected from the plurality of electrodes;
wherein the instructions for the processor determines the position information based on measured impedances between the pairs of electrodes that best fit the mathematical model.

59. The apparatus according to claim 58, wherein the position information comprises angular rotation information of the first lead and/or the second lead.

60. The apparatus according to claim 59, wherein the position information comprises angular rotation information of the first lead and the second lead.

61. The apparatus according to claim 58, wherein the position information comprises the position of the first lead and/or the second lead relative to the patient's anatomy.

62. The apparatus according to claim 58, wherein the position information comprises the position of the first lead relative to the position of the second lead.

63. The apparatus according to claim 58, wherein the position information comprises the position of the first lead relative to the patient's anatomy at a first instance of time as compared to the position of the first lead relative to the patient's anatomy at a second instance of time, and wherein the second instance of time is previous to the first instance in time.

64. The apparatus according to claim 58, wherein the position information comprises the position of the first lead relative to the second lead at a first instance of time as compared to the position of the first lead relative to the second lead at a second instance of time, and wherein the second instance of time is previous to the first instance in time.

65. The apparatus according to claim 58, wherein the delivery device further comprises a power supply, a controller, and a housing surrounding the power supply and the controller, and wherein the first lead and/or the second lead is attachable to the housing during a clinical procedure in which the delivery device is implanted in the patient.

66. The apparatus according to claim 58, wherein the instructions for the processor to determine the position information is based on data gathered prior to implantation of the delivery device in the patient.

67. The apparatus according to claim 66, wherein the data is gathered during the manufacturing of the delivery device.

68. The apparatus according to claim 58, wherein the instructions for the processor to determine the position information comprises instructions to determine a relative position between the first lead and the second lead by:
(a) measuring the impedance between at least one pair of electrodes of the first set of electrodes and at least one pair of electrodes of the second set of electrodes;
(b) fitting a curve to the measured impedances to obtain a function of the impedance to distance: Z=f(d), based on the known distances between the electrodes of each pair;
(c) measuring the impedance between at least one cross-lead pair of electrodes, each cross-lead pair comprising one electrode of the first set of electrodes and one electrode of the second set of electrodes;
(d) determining the distance between the at least one cross-lead pair of electrodes using the function of (b); and
(e) determining the relative positions of the first lead and the second lead using the calculated distances.

69. The apparatus according to claim 68, wherein the at least one pair of electrodes of the first set of electrodes comprises all pairs of electrodes of the first set of electrodes, and wherein the at least one pair of electrodes of the second set of electrodes comprises all pairs of electrodes of the second set of electrodes.

70. The apparatus according to claim 68, wherein the impedance measurements include at least 56 impedance measurements per lead.

71. The apparatus according to claim 68, wherein the at least one cross-lead pair of electrodes comprises at least 64 pairs of electrodes.

72. The apparatus according to claim 68, wherein the relative position includes a first linear offset Lx, a second linear offset Ly, and/or an angle θ between the first lead and the second lead.

73. The apparatus according to claim 58, wherein the instructions for the processor to determine the position information comprises instructions to determine a relative position between the first lead and the second lead by:
(a) measuring the impedance between at least one pair of electrodes of the first set of electrodes and at least one pair of electrodes of the second set of electrodes;
(b) creating a first resistivity profile of tissue surrounding the first lead and creating a second resistivity profile of tissue surrounding the second lead based on the impedance measurements;
(c) measuring the impedance between at least one cross-lead pair of electrodes, each cross-lead pair comprising one electrode of the first set of electrodes and one electrode of the second set of electrodes;
(d) determining the distance between the at least one cross-lead pair of electrodes using a linear resistivity assumption based on the first resistivity profile and the second resistivity profile; and
(e) determining the relative positions of the first lead and the second lead using the calculated distances.

74. The apparatus according to claim 73, wherein the at least one pair of electrodes of the first set of electrodes comprises all pairs of electrodes of the first set of electrodes, and wherein the at least one pair of electrodes of the second set of electrodes comprises all pairs of electrodes of the second set of electrodes.

75. The apparatus according to claim 73, wherein the impedance measurements include at least 56 impedance measurements per lead.

76. The apparatus according to claim 73, wherein the at least one cross-lead pair of electrodes comprises at least 64 pairs of electrodes.

77. The apparatus according to claim 73, wherein the relative position includes a first linear offset Lx, a second linear offset Ly, and/or an angle θ between the first lead and the second lead.

78. The apparatus according to claim 58, wherein the instructions for the processor further comprise instructions to characterize a migration of the first lead and/or second lead by:
(a) determining the relative positions of the first lead and the second lead at a first time T1;
(b) creating an initial graph based on the relative positions at the first time T1;
(c) determining the relative positions of the first lead and the second lead at a second time T2;
(d) creating a subsequent graph based on the relative positions at the second time T2; and
(e) determining the difference between the initial graph and the subsequent graph to determine the migration of the first lead and/or the second lead between the first time T1 and the second time T2.

79. The apparatus according to claim 78, wherein the relative positions of the first lead and the second lead are determined using a resistivity profile.

80. The apparatus according to claim 78, wherein the relative positions of the first lead and the second lead are determined using impedance measurements.

81. The apparatus according to claim 78, wherein the migration of the first lead and the second lead comprises a relative linear migration between the first lead and the second lead.

82. The apparatus according to claim 58, wherein the instructions for the processor further comprise instructions for applying one or more equations comprising the measured impedances, and wherein the instructions for the processor to determine the position information comprises instructions to determine a relative position between the first lead and second lead by:
(a) measuring the impedance between at least one pair of electrodes of the first set of electrodes and at least one pair of electrodes of the second set of electrodes;
(b) measuring the impedance between at least one cross-lead pair of electrodes, each cross-lead pair comprising one electrode of the first set of electrodes and one electrode of the second set of electrodes; and
(c) determining resistivities of layers of the body, a bias impedance, and the relative position between the first lead and second lead so as to minimize errors in the one or more equations comprising the measured impedances.

83. The apparatus according to claim 82, wherein each equation of the one or more equations equates a measured impedance to the sum of the bias impedance and a compound term.

84. The apparatus according to claim 83, wherein the compound term is a sum of a plurality of products, and wherein each product in the plurality of products comprises a resistivity of one layer of the body, a length of a line segment, and a weight.

85. The apparatus according to claim 84, wherein the line segment is the intersection of a line connecting the pair of electrodes across which the measured impedance is measured and the layer of the body.

86. The apparatus according to claim 84, wherein the weight is calculated using a weighing function of length.

87. The apparatus according to claim 82, wherein the relative position of the first lead and the second lead comprises a relative vertical displacement between the first lead and the second lead.

88. The apparatus according to claim 82, wherein the relative position of the first lead and the second lead comprises a relative horizontal displacement between the first lead and the second lead.

89. The apparatus according to claim 82, wherein the relative position of the first lead and the second lead comprises a relative angular displacement between the first lead and the second lead.
